(19) United States
(12) Patent Application Publication
(10) Pub. No.: US 2021/0236694 A1
(43) Pub. Date: Aug. 5, 2021

(54) INDUCTION OF ANTI-TUMORAL IMMUNE MICROENVIRONMENTS

(71) Applicant: The Johns Hopkins University, Baltimore, MD (US)

(72) Inventors: Jennifer H. Elisseeff, Baltimore, MD (US); Matthew T. Wolf, Baltimore, MD (US)

(21) Appl. No.: 17/052,083

(22) PCT Filed: May 3, 2018
(86) PCT No.: PCT/US2019/030618

§ 371 Date: Oct. 30, 2020

Related U.S. Application Data
(60) Provisional application No. 62/666,617, filed on May 3, 2018.

(51) Int. Cl. A61L 27/36; A61L 27/54; C07K 16/28; A61K 35/13; A61K 35/17; C07K 14/73; A61K 35/28; A61K 39/00; A61P 35/00; A61L 27/38

(52) U.S. Cl. CPC A61L 27/3633; A61K 2039/505; C07K 16/2827; C07K 16/2818; A61K 35/13; A61K 35/17; C07K 14/70514; A61K 35/28; A61K 39/0011; A61P 35/00; A61L 27/3804; A61L 27/3834; A61L 2300/426; A61K 2039/5152; A61L 27/54

(57) ABSTRACT

Implanted extracellular matrix scaffolds used for regenerative medicine generate a highly activated and unique immune response that inhibits tumor formation by T helper cell-macrophage interactions.

### CROSS-REFERENCE TO RELATED APPLICATIONS

This application claims the benefit of priority under 35 U.S.C. § 119(e) to U.S. Provisional Application No. 62/666,617, filed on May 3, 2018, which is incorporated herein by reference in its entirety.

### FIELD OF THE INVENTION

Embodiments of the invention are directed to compositions for treating cancer by inducing an immune response. In particular the compositions comprise biocompatible scaffolds.

### BACKGROUND

Regenerative medicine therapies often induce in vivo responses that are reminiscent of processes that occur in wound healing and developmental biology with the goal of restoring tissues lost due to injury or disease. Extracellular matrix (ECM) materials are acellular tissue derived scaffolds that have seen decades of clinical use in various tissue repair and regenerative medicine applications (1). ECM scaffolds facilitate several pro-regenerative processes including cell proliferation, angiogenesis, stem cell recruitment, and non-destructive Type 2 inflammation (1-4). When ECM scaffolds are implanted in areas of tissue damage, a cascade of immune and site specific stromal/progenitor cells participate in constructively remodeling the scaffold to produce site appropriate host tissue rather than scar tissue formation which often occurs with traditional synthetic polymers (5). The regenerative potential coupled with the acellular “off the shelf” nature of ECM materials makes them among the most promising for clinical tissue regeneration. While favorable to tissue repair, these pro-regenerative processes are known to play prominent roles in the formation and progression of solid tumors (6).

### SUMMARY

This Summary is provided to briefly indicate the nature and substance of the invention. It is submitted with the understanding that it will not be used to interpret or limit the scope or meaning of the claims.

In one aspect of the invention, a composition comprises a biocompatible scaffold, wherein the scaffold is pro-regenerative and comprises a biocompatible synthetic material, a biomaterial(s), an extracellular matrix or combinations thereof. In certain embodiments, the biocompatible scaffold comprises an extracellular matrix. In certain embodiments, the biocompatible scaffold is urinary bladder matrix (UBM). In certain embodiments, the biocompatible scaffold further comprises one or more immune cell modulating agents and/or cells. The immune cell modulating agents comprise: cytokines, monokines, chemokines, checkpoint agents, adjuvants, vaccines, antigens, chemotherapeutic agents or combinations thereof. In certain embodiments, the checkpoint agent comprises an inhibitor of programmed death-ligand 1 (PD-L1), programmed cell death protein 1 (PD-1) and/or cytotoxic T-lymphocyte-associated antigen 4 (CTLA4). In certain embodiments, the biocompatible scaffold further comprises stem cells, T cells, antigen presenting cells, chimeric antigen T (CAR-T) cells, CAR natural killer cells (CAR-NK), bone marrow cells or combinations thereof.

In another aspect, a method of preventing or treating cancer in a subject, comprising administering to the subject a biocompatible scaffold, wherein the biocompatible scaffold is pro-regenerative and recruits myeloid and lymphoid cells. In certain embodiments, the biocompatible scaffold optionally comprises tumor cells or cell membranes fragments thereof. The tumor cells are replication deficient. For example, the tumor cells have been obtained from the subject and can be irradiated or chemically treated to inhibit replication.

In certain embodiments, the method further comprises administering to the subject CD4⁺ T cells. The CD4⁺ T cells are autologous, haploidentical, or combinations thereof. In certain embodiments, the method optionally comprises administering to the subject stem cells, chimeric antigen T (CAR-T) cells, CAR natural killer cells (CAR-NK), bone marrow cells or combinations thereof. In certain embodiments, the method includes administering to the subject one or more chemotherapeutic agents in combination with the biocompatible scaffold.

In another aspect, a method of inducing adaptive immunity in a subject in need thereof, comprises administering to the subject a biocompatible scaffold, wherein the biocompatible scaffold comprises a biocompatible synthetic material, a biomaterial(s), an extracellular matrix or combinations thereof. The biocompatible scaffold recruits myeloid and lymphoid cells. In certain embodiments, the lymphoid cells comprise CD4⁺/CD44⁺ T cells, B220⁺ B cells, NK1.1⁺CD3⁻ natural killer cells (NK cells), NK1.1⁺/CD3⁺ NK T cells or combinations thereof. In certain embodiments, the myeloid cells comprise CD45⁺CD11b⁺ cells. In certain embodiments, the myeloid cells comprise F4/80⁺ macrophages having an M2 polarization as measured by CD206 expression.

In certain embodiments, a vaccine comprises micronized tissues and at least one of soluble tumor cell antigens, membrane-bound tumor antigens, replication deficient tumor cells, tumor cell tissue fragments or combinations thereof. In certain aspects the micronized tissues are decellularized. In certain embodiments, the micronized tissues are tissues obtained from one or more organs. In these and other embodiments, the replication deficient tumor cells are irradiated or chemically treated. In these and other embodiments, the replication deficient tumor cells, tumor cell tissue fragments are autologous, allogeneic, haplotype matched, haplotype mismatched, haplo-identical, xenogeneic, cell lines or combinations thereof. In certain embodiments, the replication deficient tumor cells, tumor cell tissue fragments are autologous. In these and other embodiments, the vaccine further comprises one or more immune cell modulating agents. In these and other embodiments, the immune cell modulating agents comprise: cytokines, monokines, chemokines, immune checkpoint inhibitors, adjuvants, vaccines, antigens, chemotherapeutic agents or combinations thereof. In certain embodiments, the immune checkpoint inhibitor is an inhibitor of programmed death-ligand 1 (PD-L1), programmed cell death protein 1 (PD-1) and/or cytotoxic T-lymphocyte-associated antigen 4 (CTLA4).

In certain embodiments, a method of treating cancer comprises administering to a subject in need thereof, a pharmaceutical composition comprising a therapeutically effective amount of the vaccines embodied herein.

Other aspects are described infra.

### Definitions

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

As used herein, the term “agent” is meant to encompass any molecule, chemical entity, composition, drug, therapeutic agent, chemotherapeutic agent, or biological agent capable of preventing, ameliorating, or treating a disease or other medical condition. The term includes small molecule compounds, antisense oligonucleotides, siRNA reagents, antibodies, antibody fragments bearing epitope recognition sites, such as Fab, Fab′, F(ab′)₂ fragments, Fv fragments, single chain antibodies, antibody mimetics (such as DARPins, affibody molecules, affilins, affitins, anticalins, avimers, fynomers, Kunitz domain peptides and monobodies), peptoids, aptamers; enzymes, peptides organic or inorganic molecules, natural or synthetic compounds and the like. An agent can be assayed in accordance with the methods of the invention at any stage during clinical trials, during pre-trial testing, or following FDA-approval.

By “ameliorate” is meant decrease, suppress, attenuate, diminish, arrest, or stabilize the development or progression of a disease.

The term “chimeric antigen receptor” or “CAR” as used herein refers to an antigen-binding domain that is fused to an intracellular signaling domain capable of activating or stimulating an immune cell, and in certain embodiments, the CAR also comprises a transmembrane domain. In certain embodiments the CAR's extracellular antigen-binding domain is composed of a single chain variable fragment (scFv) derived from fusing the variable heavy and light regions of a murine or humanized monoclonal antibody. Alternatively, scFvs may be used that are derived from Fab's (instead of from an antibody, e.g., obtained from Fab libraries). In various embodiments, the scFv is fused to the transmembrane domain and then to the intracellular signaling domain. “First-generation” CARs include those that solely provide CD3ζ signals upon antigen binding, “Second-generation”CARs include those that provide both co-stimulation (e.g., CD28 or CD137) and activation (CD3ζ). “Third-generation” CARs include those that provide multiple co-stimulation (e.g. CD28 and CD137) and activation (CD3ζ). A fourth generation of CARs have been described, CAR T cells redirected for cytokine killing (TRUCKS) where the vector containing the CAR construct possesses a cytokine cassette. When the CAR is ligated, the CAR T cell deposits a pro-inflammatory cytokine into the tumor lesion. A CAR-T cell is a T cell that expresses a chimeric antigen receptor. The phrase “chimeric antigen receptor (CAR),” as used herein and generally used in the art, refers to a recombinant fusion protein that has an antigen-specific extracellular domain coupled to an intracellular domain that directs the cell to perform a specialized function upon binding of an antigen to the extracellular domain. The terms “artificial T-cell receptor,” “chimeric T-cell receptor,” and “chimeric immunoreceptor” may each be used interchangeably herein with the term “chimeric antigen receptor.” Chimeric antigen receptors are distinguished from other antigen binding agents by their ability to both bind MHC-independent antigen and transduce activation signals via their intracellular domain.

As used herein, the term “cancer therapy” refers to a therapy useful in treating cancer. Examples of anti-cancer therapeutic agents include, but are not limited to, antibacterial agents as described herein as well as, e.g., surgery, chemotherapeutic agents, immunotherapy, growth inhibitory agents, cytotoxic agents, agents used in radiation therapy, anti-angiogenesis agents, apoptotic agents, anti-tubulin agents, and other agents to treat cancer, such as anti-HER-2 antibodies (e.g., HERCEPTIN™), anti-CD20 antibodies, an epidermal growth factor receptor (EGFR) antagonist (e.g., a tyrosine kinase inhibitor), HER1/EGFR inhibitor (e.g., erlotinib (TARCEVA™)), platelet derived growth factor inhibitors (e.g., GLEEVEC™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also contemplated for use with the methods described herein.

A “chemotherapeutic agent” is a chemical compound useful in the treatment of cancer. Examples of chemotherapeutic agents include Erlotinib (TARCEVA™, Genentech/OSI Pharm.), Bortezomib (VELCADE™, Millennium Pharm.), Fulvestrant (FASLODEX™, Astrazeneca), Sutent (SU11248, Pfizer), Letrozole (FEMARA™, Novartis), Imatinib mesylate (GLEEVEC™, Novartis), PTK787/ZK 222584 (Novartis), Oxaliplatin (Eloxatin™, Sanofi), 5-FU (5-fluorouracil), Leucovorin, Rapamycin (Sirolimus, RAPAMUNE™, Wyeth), Lapatinib (GSK572016, GlaxoSmithKline), Lonafarnib (SCH 66336), Sorafenib (BAY43-9006, Bayer Labs.), and Gefitinib (IRESSA™, Astrazeneca), AG1478, AG1571 (SU 5271; Sugen), alkylating agents such as Thiotepa and CYTOXAN™ cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analogue topotecan); bryostatin; callystatin; CC-1065 (including its adozcicsin, carzcicsin and bizcicsin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ1 and calicheamicin omega 1 (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, anthramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN™ doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, strcptonigrin, strcptozocin, tubcrcidin, ubenimcx, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacytidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK™ polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosinc; arabinoside (“Ara-C”); cyclophosphamidc; thiotcpa; taxoids, e.g., TAXOL™ paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE™ doxetaxel (Rhone-Poulenc Rorer, Antony, France); chloranbucil; GEMZAR™ gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE™ vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

As used herein, the term “chemokine” refers to soluble factors (e.g., cytokines) that have the ability to selectively induce chemotaxis and activation of leukocytes. They also trigger processes of angiogenesis, inflammation, wound healing, and tumorigenesis. Examples of chemokines include IL-8, a human homolog of murine keratinocyte chemoattractant (KC).

Also included in this definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX™ (tamoxifen)), raloxifene, droloxifene, 4-hydroxytamoxifen, trioxifene, keoxifene, LY117018, onapristone, and FARESTON™ (toremifene); (ii) aromatase inhibitors that inhibit the enzyme aromatase, which regulates estrogen production in the adrenal glands, such as, for example, 4(5)-imidazoles, aminoglutethimide, MEGASE™ (megestrol acetate), AROMASIN™ (exemestane), formestanie, fadrozole, RIVISOR™(vorozole), FEMARA™ (letrozole), and ARIMIDEX™ (anastrozole); (iii) anti-androgens such as flutamide, nilutamide, bicalutamide, leuprolide, and goserelin; as well as troxacitabine (a 1,3-dioxolane nucleoside cytosine analog); (iv) aromatase inhibitors; (v) protein kinase inhibitors; (vi) lipid kinase inhibitors; (vii) antisense oligonucleotides, particularly those which inhibit expression of genes in signaling pathways implicated in aberrant cell proliferation, such as, for example, PKC-alpha, Ralf and H-Ras; (viii) ribozymes such as a VEGF expression inhibitor (e.g., ANGIOZYME™ (ribozyme)) and a HER2 expression inhibitor; (ix) vaccines such as gene therapy vaccines, for example, ALLOVECTIN™ vaccine, LEUVECTIN™ vaccine, and VAXID™ vaccine; PROLEUKIN™ rIL-2; LURTOTECAN™ topoisomerase 1 inhibitor; ABARELIX™ rmRH; (x) anti-angiogenic agents such as bevacizumab (AVASTIN™, Genentech); and (xi) pharmaceutically acceptable salts, acids or derivatives of any of the above.

The term “combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

As used herein, the term “cytokine” refers generically to proteins released by one cell population that act on another cell as intercellular mediators or have an autocrine effect on the cells producing the proteins. Examples of such cytokines include lymphokines, monokines; interleukins (“ILs”) such as IL-1, IL-1α, IL-2, IL-3, IL-4, IL-5, IL-6, IL-7, IL-8, IL-9, IL-10, IL-11, IL-12, IL-13, IL-15, IL-17A-F, IL-18 to IL-29 (such as IL-23), IL-31, including PROLEUKIN™ rIL-2; a tumor-necrosis factor such as TNF-α or TNF-β, TGF-β1-3; and other polypeptide factors including leukemia inhibitory factor (“LIF”), ciliary neurotrophic factor (“CNTF”), CNTF-like cytokine (“CLC”), cardiotrophin (“CT”), and kit ligand (“KL”).

As used herein, the term “immune cells” generally includes white blood cells (leukocytes) which are derived from hematopoietic stem cells (HSC) produced in the bone marrow “Immune cells” includes, e.g., lymphocytes (T cells, B cells, natural killer (NK) cells) and myeloid-derived cells (neutrophil, eosinophil, basophil, monocyte, macrophage, dendritic cells).

As used herein, the term “immune checkpoint modulator” refers to an agent that interacts directly or indirectly with an immune checkpoint. In some embodiments, an immune checkpoint modulator increases an immune effector response (e.g., cytotoxic T cell response), for example by stimulating a positive signal for T cell activation. In some embodiments, an immune checkpoint modulator increases an immune effector response (e.g., cytotoxic T cell response), for example by inhibiting a negative signal for T cell activation (e.g. disinhibition). In some embodiments, an immune checkpoint modulator interferes with a signal for T cell anergy. In some embodiments, an immune checkpoint modulator reduces, removes, or prevents immune tolerance to one or more antigens.

As used herein, the term “in combination” in the context of the administration of a therapy to a subject refers to the use of more than one therapy for therapeutic benefit. The term “in combination” in the context of the administration can also refer to the prophylactic use of a therapy to a subject when used with at least one additional therapy. The use of the term “in combination” does not restrict the order in which the therapies (e.g., a first and second therapy) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject which had, has, or is susceptible to cancer. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

As used herein, “modulating” refers to an increase or decrease in an adaptive immune system response. In a preferred embodiment, this relates to an increased, up-regulated or enhanced adaptive immune system response. An effective amount of an immunomodulatory agent is an amount that when applied or administered in accordance to the techniques herein is sufficient to modulate, preferably up-regulate, an adaptive immune system response.

As used in this specification and the appended claims, the term “or” is generally employed in its sense including “and/or” unless the content clearly dictates otherwise.

The phrase “pharmaceutically acceptable carrier” refers to a carrier for the administration of a therapeutic agent. Exemplary carriers include saline, buffered saline, dextrose, water, glycerol, ethanol, and combinations thereof. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

As used herein, the terms prognostic and predictive information are used interchangeably to refer to any information that may be used to indicate any aspect of the course of a disease or condition either in the absence or presence of treatment. Such information may include, but is not limited to, the average life expectancy of a patient, the likelihood that a patient will survive for a given amount of time (e.g., 6 months, 1 year, 5 years, etc.), the likelihood that a patient will be cured of a disease, the likelihood that a patient's disease will respond to a particular therapy (wherein response may be defined in any of a variety of ways). Prognostic and predictive information are included within the broad category of diagnostic information.

By “proliferative disease” or “cancer” as used herein is meant, a disease, condition, trait, genotype or phenotype characterized by unregulated cell growth or replication as is known in the art; including colorectal cancer, as well as, for example, leukemias, e.g., acute myelogenous leukemia (AML), chronic myelogenous leukemia (CML), acute lymphocytic leukemia (ALL), and chronic lymphocytic leukemia, AIDS related cancers such as Kaposi's sarcoma; breast cancers; bone cancers such as Osteosarcoma, Chondrosarcomas, Ewing's sarcoma, Fibrosarcomas, Giant cell tumors, Adamantinomas, and Chordomas; Brain cancers such as Meningiomas, Glioblastomas, Lower-Grade Astrocytomas, Oligodendrocytomas, Pituitary Tumors, Schwannomas, and Metastatic brain cancers; cancers of the head and neck including various lymphomas such as mantle cell lymphoma, non-Hodgkins lymphoma, adenoma, squamous cell carcinoma, laryngeal carcinoma, gallbladder and bile duct cancers, cancers of the retina such as retinoblastoma, cancers of the esophagus, gastric cancers, multiple myeloma, ovarian cancer, uterine cancer, thyroid cancer, testicular cancer, endometrial cancer, melanoma, lung cancer, bladder cancer, prostate cancer, lung cancer (including non-small cell lung carcinoma), pancreatic cancer, sarcomas, Wilms' tumor, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, multidrug resistant cancers; and proliferative diseases and conditions, such as neovascularization associated with tumor angiogenesis, macular degeneration (e.g., wet/dry AMD), corneal neovascularization, diabetic retinopathy, neovascular glaucoma, myopic degeneration and other proliferative diseases and conditions such as restenosis and polycystic kidney disease, and other cancer or proliferative disease, condition, trait, genotype or phenotype that can respond to the modulation of its environment (e.g., treating the environment with an antibiotic effective against a bacterial bioform), alone or in combination with other therapies.

The term “sample” as used herein refers to a biological sample obtained for the purpose of evaluation in vitro. With regard to the methods disclosed herein, the sample or patient sample preferably may comprise any fluid or tissue. In some embodiments, the bodily fluid includes, but is not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of a blood sample, a plasma sample, a serum sample, and a urine sample. In exemplary aspects, the sample comprises blood or a fraction thereof (e.g., plasma, serum, fraction obtained via leukopheresis). Preferred samples are whole blood, serum, plasma, or urine. A sample can also be a partially purified fraction of a tissue or bodily fluid.

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

“Pharmaceutical agent,” also referred to as a “drug,” or “therapeutic agent” is used herein to refer to an agent that is administered to a subject to treat a disease, disorder, or other clinically recognized condition that is harmful to the subject, or for prophylactic purposes, and has a clinically significant effect on the body to treat or prevent the disease, disorder, or condition. Therapeutic agents include, without limitation, agents listed in the United States Pharmacopeia (USP), Goodman and Gilman's The Pharmacological Basis of Therapeutics, 12th Ed., McGraw Hill, 2001; Katzung, B. (ed.) Basic and Clinical Pharmacology, McGraw-Hill/Appleton & Lange; 8^{th} edition (Sep. 21, 2000); Physician's Desk Reference (Thomson Publishing), and/or The Merck Manual of Diagnosis and Therapy, 18^{th} ed. (2006), or the 19th ed (2011), Robert S. Porter, MD., Editor-in-chief and Justin L. Kaplan, MD., Senior Assistant Editor (eds.), Merck Publishing Group, or, in the case of animals, The Merck Veterinary Manual, 10^{th} ed., Cynthia M. Kahn, B. A., M. A. (ed.), Merck Publishing Group, 2010.

The terms “prevent”, “preventing”, “prevention”, “prophylactic treatment” and the like refer to the administration of an agent or composition to a clinically asymptomatic individual who is at risk of developing, susceptible, or predisposed to a particular adverse condition, disorder, or disease, and thus relates to the prevention of the occurrence of symptoms and/or their underlying cause.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Any genes, gene names, gene products or peptides disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes disclosed herein, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes, nucleic acid sequences or peptides are human.

### BRIEF DESCRIPTION OF THE DRAWINGS

FIGS. 1A-1I show the injectable, tissue derived urinary bladder extracellular matrix (UBM) particles inhibit tumor formation in a CD4 T helper cell dependent manner FIG. 1A: Micronized decellularized bladder (UBM) has a sheet like appearance via scanning electron microscopy (SEM Top View and Side View). UBM particles were hydrated and injected with cancer cell lines into mice to monitor the effect on tumor formation. Tumor volume and survival in (FIG. 1B) B16-F10 melanoma (C57BL/6 mice, N=5) and (FIG. 1C) CT26 colon carcinoma (balb/c mice, N=5). FIG. 1D: Bioluminescent imaging of Luciferase transduced B16-F10 melanoma cells 1-5 days after implantation with Saline or UBM. FIG. 1E: Macroscopic and histologic appearance of B16-F10 tumors 14 days following implantation with Saline or UBM (H&E stain, 200× or 50× mosaic images). Tumor nodules are denoted by arrowheads (50× mosaics) and by “Tu” (200× images). UBM is denoted by dashed line and “UBM” label. FIG. 1F: Immunofluorescent labeling for CD3⁺ T cells (red) and B220+B cells (green) with DAPI counterstain (blue). Representative of N=3 animals. FIG. 1G: The proportion of CD4⁺ and CD8⁺ T cells isolated 7 days following B16-10 delivery with UBM or Saline was determined with flow cytometry (N=5, mean±SE). FIG. 1H: FoxP3⁺ expression in CD4⁺ cells isolated from tumor or draining lymph node (DLN) 7 days following Saline or UBM delivery (N=5, mean±SE). Representative flow cytometry plots with the UBM FMO isotype control. FIG. 1I: B16-F10 tumor growth in WT, lymphocyte deficient Rag1^{-/-}, and CD4⁺ T cell repopulated Rag1^{-/-} C57BL/6 mice following delivery with UBM or Saline for tumor volume measurements and survival (N=5, mean±SE). (Statistics) Flow cytometry: * P<0.05, *** P<0.001, student's t-test of Saline vs UBM. Tumor volume: * P<0.05 WT Saline vs WT UBM, P<0.05 for Rag1^{-/-} UBM vs Rag1^{-/-}+CD4 UBM, two-way repeated measures ANOVA with post-hoc Tukey test at each time point before sacrifice. Survival: * P<0.05, log-rank test of each group compared to WT saline with the Sidak correction (significance indicated in legend).

FIGS. 2A-2F show the immunophenotyping of T cells isolated from UBM delivered B16-F10 tumors. CD3⁺ T cells were sorted for multiplex gene expression analysis using the NanoString platform. FIG. 2A: Volcano plot of genes differentially regulated in UBM derived T cells compared to Saline at 14 days. Significant regulation was determined from false discovery rate adjusted p-values. FIG. 2B: Normalized counts of T_{H}2 associated (Il4, Il13) and myeloid regulating (Csf1, Cd40lg) gene transcripts after 7, 14, and 21 days. FIG. 2C: Differential expression T_{H}2 associated, T cell activation, and NKT cell related gene sets in UBM relative to Saline B16-F10 tumor T cells at 14 days. FIG. 2D: Intracellular cytokine staining of IL4 and IFNγ in Saline and UBM delivered F16-F10 tumors after 14 days compared to FMO controls. (N=5, mean±SE). * P<0.05, student's t-test of Saline vs UBM. FIG. 2E: T cell (CD3⁺NK1.1⁻), NKT cell (CD3⁺NK1.1⁺) and NK cell (CD3⁻NK1.1⁺) density (cells per mm³) after 14 days (N=5, mean±SE). FIG. 2F: Effect of exogenously delivered IL4-complex delivery on B16-F10 tumor formation. Saline and UBM B16-F10 delivery was compared to delivery in combination with 10 μg/injection IL4-complex (IL4c), a stabilized formulation of recombinant IL4. Average tumor growth and survival were monitored (N=5, mean±SE). (Statistics) Flow cytometry: * P<0.05, student's t-test of Saline vs UBM. Tumor volume: ‡P<0.05 for Saline vs UBM, Saline+IL4c, UBM+IL4c, two-way repeated measures ANOVA with post-hoc Tukey test at each time point before sacrifice. Survival: * P<0.05, ** P<0.01, log-rank test compared to WT saline with the Sidak correction (significant indicated in legend).

FIGS. 3A-3G show the lymphocyte dependent myeloid cell recruitment and M2 macrophage polarization in UBM and Saline delivered B16-F10 tumors. FIG. 3A: SEM shows substantial cell infiltration in and around acellular UBM after implantation in WT mice. FIG. 3B: Flow cytometry analysis shows the majority of viable CD45⁺ cells are CD11b⁺ myeloid cells. FIG. 3C: The number of myeloid cells recruited to UBM decreases lymphocyte deficient Rag1^{-/-} mice (N=5, mean±SE). FIG. 3D: Flow cytometry plots and myeloid gating strategy of Saline and UBM delivered B16-F10 tumors in WT and Rag1^{-/-} C57BL/6 mice. FIG. 3E: Myeloid quantification of eosinophil, neutrophil, and monocyte infiltration in WT and Rag1^{-/-} mice determined from flow cytometry (saline delivered tumors were pooled from 5 animals, N=5 for UBM delivered cells, mean±SE). FIG. 3F: Quantification of the mean fluorescent intensity of macrophage polarization markers CD206 (M2) and CD86 (M1) in CD11c and F4/80 expressing sub-populations determined from flow cytometry. (Saline delivered tumors were pooled from 5 animals, N=5 for UBM delivered cells, mean±SE). FIG. 3G: B16-F10 tumor growth following macrophage ablation. Mice were injected with clodronate liposomes (Clod^{Lipo}) to deplete circulating macrophage progenitors or control PBS liposomes (PBS^{Lipo}) before and maintained during B16-F10 tumor growth. B16-F10 tumor volume and survival was monitored (N=5 for PBS^{Lipo} groups and N=3 for Clod^{Lipo} groups, mean±SE). (Statistics) * P<0.05, ** P<0.01, *** P<0.001 student's t-test of WT UBM vs Rag1^{-/-} UBM. Tumor volume: two-way repeated measures ANOVA with post-hoc Tukey test at each time point before sacrifice. # P<0.05 for Saline+PBS^{Lipo} vs UBM+PBS^{Lipo}, Saline+PBS^{Lipo} vs. Saline+Clod^{Lipo} and UBM+PBS^{Lipo} vs UBM+Clod^{Lipo}.

FIGS. 4A-4D show the immunophenotyping of macrophages isolated from UBM delivered B16-F10 tumors. CD11b⁺F4/80⁺ cells were sorted for gene expression analysis using the NanoString platform. FIG. 4A: Volcano plot of genes differentially regulated in UBM derived macrophages compared to Saline at 14 days. Significant regulation was determined from false discovery rate adjusted p-values. FIG. 4B: Average normalized counts of M2 associated (Arg1, Mrc1) and M1 associated (Cd86, Cd80) gene transcripts after 7, 14, and 21 days (N=3-4 except 7 day Saline which was pooled from 3 animals, mean±SD). FIG. 4C: Differential expression of M1 and M2 associated genes in UBM relative to Saline B16-F10 tumor macrophages at 14 days. FIG. 4D: Differential fold changes of complement, angiogenesis, and cell regulation genes in UBM relative to Saline B16-F10 tumor macrophages at 14 days (N=4, mean±SE).

FIGS. 5A-5E show the effect of synthetic particles on B16-F10 tumor formation and myeloid cell recruitment. Saline and UBM particulate delivery of B16-F10 cells was compared to the synthetic particulates Aluminum hydroxide (Alum) and mesoporous silica (Silica) in WT and Rag1^{-/-} C57BL/6 mice. FIG. 5A: Average tumor volume and (FIG. 5B) survival were monitored (N=5, mean±SE). FIG. 5C: Concatenated flow cytometry plots of myeloid cells isolated from Saline (N=3) and Alum (N=5) after 7 days post injection with B16-F10 cells. FIG. 5D: Average eosinophil, granulocyte, and monocyte infiltration as % of CD11b cells in Saline, Alum, and Silica delivered B16-F10 cells after 7 days (N=3 Saline, N=5 Alum and Silica, mean±SE). FIG. 5E: Average F4/80 and CD11c infiltration % of CD11b cells in Saline, Alum, and Silica delivered B16-F10 cells after 7 days (N=3 Saline, N=5 Alum and Silica, mean±SE). (Statistics) Tumor volume: P <0.05 WT saline vs WT UBM, WT Alum, WT Silica; Rag1^{-/-} saline vs Rag1^{-/-} Alum, Rag1^{-/-} Silica; WT UBM vs Rag1^{-/-} UBM; & P<0.05 WT Saline vs WT Alum and Rag1^{-/-} Alum, two-way repeated measures ANOVA with post-hoc Tukey test at each time point before sacrifice. Survival and average number of days to sacrifice: Solid line indicates mean days to sacrifice in WT Saline and the dashed line indicates mean days to sacrifice in WT UBM delivered B16-F10 cells. * P<0.05, log-rank test compared to WT saline with the Sidak correction. Flow cytometry: * P<0.05, ** P<0.01, *** P<0.001 student's t-test compared to WT Saline (significance indicated in legend).

FIGS. 6A-6E show the synergistic tumor inhibition of UBM in combination with immune checkpoint blockade immunotherapy. Saline and UBM delivery of B16-F10 cells was followed by treatment with monoclonal antibodies blocking PD-1, PD-L1, or PD-L2 as compared to isotype controls. FIG. 6A: Individual tumor growth curves comparing Saline and UBM with PD-1 treatment. FIG. 6B: Average tumor volume and (FIG. 6C) survival were monitored with checkpoint inhibitors (N=8-10, mean±SE). Arrows indicate treatment times. FIG. 6D: Individual tumor growth curves of UBM with anti-PD-1 treatment in a therapeutic model. B16-F10 cells were given a day to engraft before UBM or Saline was injected followed by anti-PD-1 injections or isotype controls 4 days later. FIG. 6E: Survival following delayed UBM implantation with anti-PD-1 treatment (N=5). (Statistics) Tumor volume: P<0.05 UBM+isotype vs UBM+PD-1 or PD-L1. **** P<0.0001 for all UBM treatments vs Saline+isotype. Two-way repeated measures ANOVA with post-hoc Tukey test at each time point before sacrifice. Survival: * P<0.05, ** P<0.01, log-rank test with the Sidak correction.

FIGS. 7A-7G show that UBM implantation does not promote tumor growth in an orthotopic breast cancer resection model. FIG. 7A: Description of model: (i) Luciferase expressing 4T1 breast carcinoma (4T1-Luc) were injected into the mammary fat pad of female balb/c mice, (ii) 4T1-Luc tumors grew to ˜1 cm in greatest dimension, (iii) tumors were resected and either UBM or Saline immediately injected in the resection bed, and (iv) tumor recurrence at the primary site and lung metastases monitored. FIG. 7B: Overlays of individual tumor growth curves at primary injection site and (FIG. 7C) survival with Saline or UBM injection. Numerals correspond with steps in FIG. 7A. FIG. 7D: Representative whole animal bioluminescence imaging of 4T1-Luc cells 1 week post resection and implantation with UBM or Saline. FIG. 7E: Bioluminescence quantification at the primary tumor site and lung metastases. (N=5, mean±SE). FIG. 7F: Individual tumor growth curves and mean tumor volume and (FIG. 7G) survival of subcutaneous delivery of 4T1 cells with UBM or Saline. (N=5, mean±SE) (Statistics) Tumor volume: * P<0.05 UBM. Two-way repeated measures ANOVA with post-hoc Tukey test at each time point before sacrifice. Survival: * P<0.05,log-rank test with the Sidak correction.

FIG. 8 shows the UBM dose response for tumor growth inhibition. UBM particles were injected at 4 different concentrations subcutaneously with B16-F10 cells. Each 100 μl injected contained either 0 mg UBM/ml (Saline), 12.5 mg UBM/ml, 25 mg UBM/ml, or 50 mg UBM/ml. Tumor growth and survival was monitored for each concentration (N=5, mean±SE).

FIG. 9 shows B16-F10 viability and adhesion to UBM in vitro. B16-F10 cells were seeded on glass coverslips coated with UBM or bovine Type I collagen for 1.5 hours. Cells were stained with Calcein-AM and counted. (N=3 coverslips, N=3 fields of view, mean±SE). Significance defined as P<0.05 student's t-test to uncoated coverslips.

FIGS. 10A-10C show the lymphocyte gating strategy in lymph nodes and B16-F10 tumors. Cells from tumor draining lymph nodes (FIG. 10A) and tumors (FIG. 10B) were isolated and stained for viability, CD45, CD19, CD3, NK1.1, CD4, CD8, CD62L, CD44 (accompanying analysis in FIGS. 2A-2F). Tumor gating strategy differed from lymph nodes to exclude B16-F10 cells from the analysis. (C) Nearly all tumor infiltrating T cells are antigen experienced (CD44⁺). (N=5, mean±SE) *P<0.05, student's T-test of Saline vs UBM.

FIGS. 11A-11C show the CD4⁺ T cell purity in adoptive transfer experiments. FIG. 11A: Flow cytometry was conducted to determine T cell purity before (unpurified) and after negative selection for CD4⁺ T cells from WT mouse lymph tissue for use in adoptive transfer experiments. FIG. 11B: Purified CD4⁺ T cells were then injected intravenously into lymphocyte deficient Rag1^{-/-} mice. Peripheral blood was collected from WT, Rag1^{-/-} mice, and CD4⁺ T cell repopulated Rag1^{-/-} mice for flow cytometry analysis for B cells (CD19), CD4 T cells (CD3/CD4), and CD8 T cells (CD3/CD8) and quantified. (N=10, mean±SE, plots are concatenated from 10 samples). FIG. 11C: B16-F10 cells were injected into WT, Rag1^{-/-} mice, and CD4 repopulated Rag1^{-/-} with and without UBM. After tumors had grown to 2 cm in size, animals were sacrificed and spleens harvested for histologic analysis. Immunofluorescent staining for CD4 and CD8 showed that CD4 repopulation was effective and pure. CD8⁺ cells in Rag1^{-/-} and CD4⁺ T cell repopulated Rag1^{-/-} mice were also CD3- and had a dendritic appearance (N=4-5, representative of 3 fields of view per sample).

FIG. 12 shows the differentially expressed genes in T cells sorted from Saline and UBM tumors. CD3⁺ cells were sorted from UBM and saline delivered tumors for multiplex gene expression analysis conducted using the NanoString platform (accompanying analysis in FIGS. 2A-2F). The top 30 fold changes in significantly regulated genes 14 days post B16-F10 injection are presented, as well as genes related to regulation of cellular processes and lineage markers.

FIGS. 13A, 13B show the clodronate liposome macrophage depletion. The effect of macrophage on UBM mediated tumor growth inhibition was evaluated by clodronate depletion. FIG. 13A: Injection schedule for clodronate or PBS control loaded liposome injections, which begin 4 days before B16-F10 melanoma implantation and continue every 2 days until sacrifice (“sac”). FIG. 13B: Flow cytometry analysis of peripheral blood to verify a reduction in the number Ly6C high macrophage progenitors (CD11b⁺Ly6G⁻) in clodronate liposome treated animals compared to PBS liposome controls.

FIG. 14 shows the differentially expressed genes in macrophages sorted from UBM and Saline tumors. F4/80⁺ cells were sorted from UBM and Saline delivered tumors for multiplex gene expression analysis conducted using the NanoString platform (accompanying analysis in FIGS. 4A-4D). The top 30 fold changes in significantly regulated genes at day 14 post B16-F10 injection are presented, as well as genes related to antigen presentation, toll like receptors (TLRs), T cell regulation, endosomal and lysosomal activity, scavenger receptors, and lipid transport.

FIG. 15 shows the gating strategy for T cell and macrophage cell sorting. T cells and macrophages were sorted UBM and Saline delivered tumors for multiplex gene expression analysis.

### DETAILED DESCRIPTION

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

The lingering uncertainty of whether regenerative strategies provide a fertile environment for de novo or recurrent tumor formation has been a barrier to clinical translation (7, 8). Accordingly, this study was directed, in part, as to how pro-regenerative scaffolds affect the initial stages of tumor formation. The tumor microenvironment is the net result of coordinated interactions between neoplastic cells and numerous support stroma that have been co-opted to enable tumor progression (9) Immune cells in particular can play opposing roles in a tumor: surveillance and clearance of cancer versus promotion of tumor growth via expression of immunosuppressive cytokines and surface ligands. Typically, immune cells that undergo a classically pro-inflammatory Type 1 skewed activation state are associated with the ability to engage in tumor clearance. Type 1 T helper cells (T_{H}1 cells), cytotoxic T cells, and Type 1 macrophages (M1 cells) are among the most frequently studied effectors of tumor killing (10). Conversely, Type 2 responses, associated with eosinophils, T_{H}2 cells, and M2 macrophages are correlated with a tumor permissive environment (11). ECM biomaterials are characterized by rapid recruitment of myeloid cells, and notably the presence of M2 polarized macrophages regulated in part by T_{H}2 T cells (4). In the examples section which follows, the results show that the T_{H}2-M2 response, similar to those observed in tumors, are promoted by the ECM biomaterials.

### Biocompatible Scaffolds

ECM scaffold materials are used in soft tissue repair applications where excessive scar tissue can be deleterious. ECM scaffolds have been prepared from numerous mammalian (allogeneic and xenogeneic) sources (12), however similarly prepared ECM materials elicit comparable functional repair outcomes in many instances (13). Clinical applications include replacement and reconstruction of tissue voids left following tumor resection; mastectomy/lumpectomy following breast cancer, dural repair after meningioma, and re-epithelialization following esophageal cancer resection (14-17). These applications potentially place ECM scaffolds in proximity to residual cancer cells near the margins, and thus a tumor permissive environment may have severe consequences. Paradoxically, prior to this study, it was unknown what effect the pro-regenerative ECM materials have on immune microenvironment has on tumor formation and progression.

A diverse population of immune cells is recruited into scaffolds and the surrounding area, including macrophages, T lymphocytes and B lymphocytes. The scaffolds induced a pro-regenerative type-2 response, characterized by an mTOR/Rictor-dependent T_{H}2 pathway and IL-4-dependent macrophage polarization, which is critical for functional muscle regeneration. Targeting the adaptive components of the immune system during the process of biomaterials design may support the development of future therapies that efficiently control immune balance in tissues, ultimately stimulating an anti-tumor response.

Generally, any material that is biocompatible, biodegradable, and has mechanical properties similar to that of native tissue can be used as a scaffold, including for example elastomeric scaffolds. In one embodiment, the scaffold comprises a powdered biological extracellular matrix (ECM). In certain embodiments, the ECM is encased in a laminar sheath of ECM. In yet another embodiment, the scaffold comprises particulate ECM derived from porcine urinary bladder (UBM-ECM).

The biocompatible scaffolds herein are pro-regenerative scaffolds which can be used for a large number of medical applications including, but not limited to, wound healing, tissue remodeling, and tissue regeneration. In one non-limiting embodiment, the scaffold is used to induce an anti-tumor immune response. In certain embodiments, the scaffold further comprises one or more immune cell modulating agents. The immune cell modulating agents comprise: cytokines, monokines, chemokines, checkpoint agents, adjuvants, vaccines, antigens, chemotherapeutic agents or combinations thereof. In certain embodiments, the checkpoint agent is an inhibitor of programmed death-ligand 1 (PD-L1), programmed cell death protein 1 (PD-1) and/or cytotoxic T-lymphocyteassociated antigen 4 (CTLA4). Examples of checkpoint inhibitors include: Pembrolizumab, Nivolumab, Atezolizumab, Avelumab. In certain embodiments, one or more checkpoint inhibitors are administered as co-therapeutic agents with other immunotherapy drugs blocking LAG3, B7-H3, KIR, OX40, PARP, CD27, and ICOS. In certain embodiments, the biocompatible scaffold comprises tumor cells or cell membranes fragments thereof. In certain embodiments, the scaffold comprises agents to recruit selected cell types, such as stem cells, or induce differentiation of cells. In certain embodiments, combinations of cells and one or more immune cell modulating agents are added to the scaffold before or during implantation in a patient.

Historical classification of macrophages defines the M1 phenotype (e.g., CD86⁺ and Nos2, Tnfa expression) and M2 phenotype (e.g., CD206⁺ and Arg1, Fizz1 expression) as opposite poles governing pro-inflammatory and anti-inflammatory or wound-healing responses, respectively. Recent evidence highlights the heterogeneity of macrophage phenotype and the role of multiple macrophage subtypes in cardiac wound healing (Epelman S., et al. Nat Rev Immunol, 2015, 15(2): p. 117-29), scar formation, and outcomes of certain cancers (Lewis, C. E. and J. W. Pollard, Cancer Res, 2006. 66(2): p. 605-12). Macrophage polarization occurs along a spectrum, and a coordinated timing of the differing phenotypes enables clearance of infection followed by healing of damaged tissue. This polarization is mediated by both environmental factors and further, can be modified by signals from cells of the adaptive immune system, particularly T cells. Macrophages and dendritic cells present antigens and activate T cells, which in turn modulate other immune cells through secretion of cytokines. One such cytokine is interleukin 4 (IL-4) (Tidball, J. G. and S. A. Villalta, Am J Physiol Regul Integr Comp Physiol, 2010. 298(5): p. R1173-87; Salmon-Ehr, V., et al., Lab Invest, 2000. 80(8): p. 1337-43).

According to the techniques herein, biomaterials may induce influx of macrophages with a particularly strong M2 phenotype and that this phenotype may be dependent on the adaptive immune system, which is characterized by a T helper 2 (T_{H}2) cell phenotype. The enhanced T_{H}2/M2 response may be associated with a pro-regenerative cytokine environment and anti-tumor responses as described in the examples section which follows.

The scaffolds can comprise any suitable combination of synthetic polymeric components and biological polymeric components. As used herein, the term “polymer” refers to both synthetic polymeric components and biological polymeric components. “Biological polymer(s)” are polymers that can be obtained from biological sources, such as, without limitation, mammalian or vertebrate tissue, as in the case of certain extracellular matrix-derived (ECM-derived) compositions. Biological polymers can be modified by additional processing steps. Polymer(s), in general include, for example and without limitation, mono-polymer(s), copolymer(s), polymeric blend(s), block polymer(s), block copolymer(s), cross-linked polymer(s), non-cross-linked polymer(s), linear-, branched-, comb-, star-, and/or dendrite-shaped polymer(s), where polymer(s) can be formed into any useful form, for example and without limitation, a hydrogel, a porous mesh, a fiber, woven mesh, or non-woven mesh, such as, for example and without limitation, a non-woven mesh formed by electrodeposition.

Generally, the polymeric components suitable for the scaffold described herein may be any polymer that is biodegradable and biocompatible. By “biodegradable”, it is meant that a polymer, once implanted and placed in contact with bodily fluids and/or tissues, will degrade either partially or completely through chemical, biochemical and/or enzymatic processes. Non-limiting examples of such chemical reactions include acid/base reactions, hydrolysis reactions, and enzymatic cleavage. In certain non-limiting embodiments, the biodegradable polymers may comprise homopolymers, copolymers, and/or polymeric blends comprising, without limitation, one or more of the following monomers: glycolide, lactide, caprolactone, dioxanone, and trimethylene carbonate. Non-limiting examples of biodegradeable polymers include poly(ester urethane) urea elastomers (PEUU) and poly(ether ester urethane) urea elastomers (PEEUU). In other non-limiting embodiments, the polymer(s) comprise labile chemical moieties, non-limiting examples of which include esters, anhydrides, polyanhydrides, or amides, which can be useful in, for example and without limitation, controlling the degradation rate of the scaffold and/or the release rate of therapeutic agents from the scaffold. Alternatively, the polymer(s) may contain peptides or biomacromolecules as building blocks which are susceptible to chemical reactions once placed in situ. For example, the polymer is a polypeptide comprising the amino acid sequence alanine-alanine-lysine, which confers enzymatic lability to the polymer. In another non-limiting embodiment, the polymer composition may comprise a biomacromolecular component derived from an ECM. For example, the polymer composition may comprise the biomacromolecule collagen so that collagenase, which is present in situ, can degrade the collagen.

In embodiments, the scaffolds are biocompatible. By “biocompatible,” it is meant that a polymer composition and its normal in vivo degradation products are cytocompatible and are substantially non-toxic and non-carcinogenic in a patient within useful, practical and/or acceptable tolerances. By “cytocompatible,” it is meant that the polymer can sustain a population of cells and/or the polymer composition, device, and degradation products, thereof are not cytotoxic and/or carcinogenic within useful, practical and/or acceptable tolerances. For example, the scaffold when placed in a human epithelial cell culture does not adversely affect the viability, growth, adhesion, and number of cells. In one non-limiting embodiment, the compositions, and/or devices are “biocompatible” to the extent they are acceptable for use in a human patient according to applicable regulatory standards in a given jurisdiction. In another example the biocompatible polymer, when implanted in a patient, does not cause a substantial adverse reaction or substantial harm to cells and tissues in the body, for instance, the polymer composition or device does not cause necrosis or an infection resulting in harm to tissues from the implanted scaffold.

The biocompatible scaffold or extracellular matrix comprises and includes an extracellular matrix-derived material. As used herein, the terms “extracellular matrix” and “ECM” refer to a complex mixture of structural and functional biomolecules and/or biomacromolecules including, but not limited to, structural proteins, specialized proteins, proteoglycans, glycosaminoglycans, and growth factors that surround and support cells within mammalian tissues and, unless otherwise indicated, is acellular. By “ECM-derived material” it is meant a composition that is prepared from a natural ECM or from an in vitro source wherein the ECM is produced by cultured cells and comprises one or more polymeric components (constituents) of native ECM. ECM preparations can be considered to be “decellularized” or “acellular”, meaning the cells have been removed from the source tissue through processes described herein and known in the art.

According to one non-limiting example of the ECM-derived material, ECM is isolated from a vertebrate animal, for example, from a warm blooded mammalian vertebrate animal including, but not limited to, human, monkey, pig, cow, sheep, etc. The ECM may be derived from any organ or tissue, including without limitation, urinary bladder, intestine, liver, heart, esophagus, spleen, stomach and dermis. The ECM can comprise any portion or tissue obtained from an organ, including, for example and without limitation, submucosa, epithelial basement membrane, tunica propria, etc. In one non-limiting embodiment, the ECM is isolated from urinary bladder, which may or may not include the basement membrane. In another non-limiting embodiment, the ECM includes at least a portion of the basement membrane. In certain non-limiting embodiments, the material that serves as the biological component of the scaffold consists primarily (e.g., greater than 70%, 80%, or 90%) of ECM. In another non-limiting embodiment, the scaffold may contain at least 50% ECM, at least 60% ECM, at least 70% ECM, and at least 80% ECM. In yet another non-limiting embodiment, the biodegradable elastomeric scaffold comprises at least 10% ECM. The ECM material may or may not retain some of the cellular elements that comprised the original tissue such as capillary endothelial cells or fibrocytes. The type of ECM used in the scaffold can vary depending on the intended immune cell or other cell types to be recruited

In one non-limiting embodiment, the ECM is harvested from porcine urinary bladders (also known as urinary bladder matrix or UBM). Briefly, the ECM is prepared by removing the urinary bladder tissue from a pig and trimming residual external connective tissues, including adipose tissue. All residual urine is removed by repeated washes with tap water. The tissue is delaminated by first soaking the tissue in a deepithelializing solution, for example and without limitation, hypertonic saline (e.g. 1.0 N saline), for periods of time ranging from ten minutes to four hours. Exposure to hypertonic saline solution removes the epithelial cells from the underlying basement membrane. Optionally, a calcium chelating agent may be added to the saline solution. The tissue remaining after the initial delamination procedure includes the epithelial basement membrane and tissue layers abluminal to the epithelial basement membrane. The relatively fragile epithelial basement membrane is invariably damaged and removed by any mechanical abrasion on the luminal surface. This tissue is next subjected to further treatment to remove most of the abluminal tissues but maintain the epithelial basement membrane and the tunica propria. The outer serosal, adventitial, tunica muscularis mucosa, tunica submucosa and most of the muscularis mucosa are removed from the remaining deepithelialized tissue by mechanical abrasion or by a combination of enzymatic treatment (e.g., using trypsin or collagenase) followed by hydration, and abrasion. Mechanical removal of these tissues is accomplished by removal of mesenteric tissues with, for example and without limitation, Adson-Brown forceps and Metzenbaum scissors and wiping away the tunica muscularis and tunica submucosa using a longitudinal wiping motion with a scalpel handle or other rigid object wrapped in moistened gauze. Automated robotic procedures involving cutting blades, lasers and other methods of tissue separation are also contemplated.

In some embodiments ECM is prepared as a powder. Such powder can be made according to the method of Gilbert et al., Biomaterials 26 (2005) 1431-1435, herein incorporated by reference in its entirety. For example, UBM sheets can be lyophilized and then chopped into small sheets for immersion in liquid nitrogen. The snap frozen material can then be comminuted so that particles are small enough to be placed in a rotary knife mill, where the ECM is powdered. Similarly, by precipitating NaCl within the ECM tissue the material will fracture into uniformly sized particles, which can be snap frozen, lyophilized, and powdered. The ECM typically is derived from mammalian tissue, such as, without limitation from one of urinary bladder, spleen, liver, heart, pancreas, ovary, or small intestine. In certain embodiments, the ECM is derived from a pig, cow, horse, monkey, or human.

In further embodiments, cells, drugs, cytokines and/or growth factors can be added to the gel prior to, during or after gelation, so long as the bioactivity of the cells, drugs, cytokines and/or growth factors is not substantially or practically (for the intended use) affected by the processing of the gel to its final form.

Micronization of Tissues: Once the tissues have been dehydrated, the dehydrated tissue(s) is micronized. The micronized compositions can be produced using instruments known in the art. For example, the Retsch Oscillating Mill MM400 can be used to produce the micronized compositions described herein. The particle size of the materials in the micronized composition can vary as well depending upon the application of the micronized composition. In one aspect, the micronized composition has particles that are less than 500 μm, less than 400 μm, less than 300 μm, or from 25 μm to 300 μm, from 25 μm to 200 μm, or from 25 μm to 150 μm. In certain aspects, particles having a larger diameter (e.g. 150 μm to 350 μm) are desirable.

In one embodiment, micronization is performed by mechanical grinding or shredding. In another aspect, micronization is performed cryogenic grinding. In this aspect, the grinding jar containing the tissue is continually cooled with liquid nitrogen from the integrated cooling system before and during the grinding process. Thus the sample is embrittled and volatile components are preserved. Moreover, the denaturing of proteins in the tissues or tissue layer,

The selection of components used to make the micronized components described herein can vary depending upon the end-use of the composition. For example, bladder, amnion, chorion, etc., or any combination thereof as individual components can be admixed with one another and subsequently micronized. In another aspect, one or more ECMs composed of one or more tissue sources.

In addition to urinary bladder tissue, additional components can be added to the composition prior to and/or after micronization. In one aspect, a filler can be added. Examples of fillers include, but are not limited to, allograft pericardium, allograft acellular dermis, Wharton's jelly separated from vascular structures (i.e., umbilical vein and artery) and surrounding membrane, purified xenograft Type-1 collagen, biocellulose polymers or copolymers, biocompatible synthetic polymer or copolymer films, purified small intestinal submucosa, bladder acellular matrix, cadaveric fascia, or any combination thereof.

In another embodiment, a bioactive agent can be added to the composition prior to and/or after micronization. Examples of bioactive agents include, but are not limited to, naturally occurring growth factors sourced from platelet concentrates, either using autologous blood collection and separation products, or platelet concentrates sourced from expired banked blood; bone marrow aspirate; stem cells derived from concentrated human placental cord blood stem cells, concentrated amniotic fluid stem cells or stem cells grown in a bioreactor; or antibiotic, immunomodulatory agents and the like. Upon application of the micronized composition with bioactive agent to the region of interest, the bioactive agent is delivered to the region over time. Thus, the micronized particles described herein are useful as delivery devices of bioactive agents and other pharmaceutical agents when administered to a subject. Release profiles can be modified based on, among other things, the selection of the components used to make the micronized compositions as well as the size of the particles.

In certain embodiments, the micronized composition can be used to form a three-dimensional construct. For example, the micronized particles can be treated with a cross-linking agent then placed in a mold having specific dimensions. Alternatively, the micronized particles can be placed into the mold and subsequently treated with the cross-linking agent. In one aspect, the cross-linked particles can be manually formed into any desired shape. In other aspects, one or more adhesives can be admixed with an adhesive prior to being introduced into the mold. Examples of such adhesives include, but are not limited to, fibrin sealants, cyanoacrylates, gelatin and thrombin products, polyethylene glycol polymer, albumin, and glutaraldehyde products. Not wishing to be bound by theory, the three-dimensional construct composed of smaller micronized particles will produce a denser product capable of bearing mechanical loads. Alternatively, larger micronized particles will produce constructs that are less dense and possess compressive properties. This feature can be useful in non-load void filling, especially where it is desirable to have a product that will conform to irregular shapes. The three-dimensional constructs can include one or more bioactive agents described herein.

In certain embodiments, the concentration of the cross-linking agent is from 0.1 M to 5 M, 0.1 M to 4 M, 0.1 M to 3 M, 0.1 M to 2 M, or 0.1 M to 1 M. The cross-linking agent generally possesses two or more functional groups capable of reacting with proteins to produce covalent bonds. In one aspect, the cross-linking agent possesses groups that can react with amino groups present on the protein. Examples of such functional groups include, but are not limited to, hydroxyl groups, substituted or unsubstituted amino groups, carboxyl groups, and aldehyde groups. In one aspect, the cross-linker can be a dialdehyde such as, for example, glutaraldehyde. In another aspect, the cross-linker can be a carbodiimide such as, for example, (N-(3-dimethylaminopropyl)-N′-ethyl-carbodiimide (EDC). In other aspects, the cross-linker can be an oxidized dextran, p-azidobenzoyl hydrazide, N-[alpha-maleimidoacetoxy]succinimide ester, p-azidophenyl glyoxal monohydrate, bis-[beta-(4-azidosalicylamido)ethyl]disulfide, bis-[sulfosuccinimidyl]suberate, dithiobis[succinimidyl]propionate, disuccinimidyl suberate, and 1-ethyl-3-[3-dimethylaminopropyl]carbodiimide hydrochloride, a bifunctional oxirane (OXR), or ethylene glycol diglycidyl ether (EGDE).

In certain embodiments, sugar is the cross-linking agent, where the sugar can react with proteins present in the ECM to form a covalent bond. For example, the sugar can react with proteins by the Maillard reaction, which is initiated by the nonenzymatic glycosylation of amino groups on proteins by reducing sugars and leads to the subsequent formation of covalent bonds. Examples of sugars useful as a cross-linking agent include, but are not limited to, D-ribose, glycerose, altrose, talose, ertheose, glucose, lyxose, mannose, xylose, gulose, arabinose, idose, allose, galactose, maltose, lactose, sucrose, cellibiose, gentibiose, melibiose, turanose, trehalose, isomaltose, or any combination thereof.

In other embodiments, the micronized compositions described herein can be formulated in any excipient the biological system or entity can tolerate to produce pharmaceutical compositions. Examples of such excipients include, but are not limited to, water, aqueous hyaluronic acid, saline, Ringer's solution, dextrose solution, Hank's solution, and other aqueous physiologically balanced salt solutions. Nonaqueous vehicles, such as fixed oils, vegetable oils such as olive oil and sesame oil, triglycerides, propylene glycol, polyethylene glycol, and injectable organic esters such as ethyl oleate can also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosol, cresols, formalin and benzyl alcohol. In certain aspects, the pH can be modified depending upon the mode of administration. Additionally, the pharmaceutical compositions can include carriers, thickeners, diluents, preservatives, surface active agents and the like in addition to the compounds described herein.

The pharmaceutical compositions can be prepared using techniques known in the art. In one aspect, the composition is prepared by admixing a micronized composition described herein with a pharmaceutically-acceptable compound and/or carrier. The term “admixing” is defined as mixing the two components together so that there is no chemical reaction or physical interaction. The term “admixing” also includes the chemical reaction or physical interaction between the compound and the pharmaceutically-acceptable compound.

It will be appreciated that the actual preferred amounts of micronized composition in a specified case will vary according to the specific compound being utilized, the particular compositions formulated, the mode of application, and the particular situs and subject being treated. Dosages for a given host can be determined using conventional considerations, e.g. by customary comparison of the differential activities of the subject compounds and of a known agent, e.g., by means of an appropriate conventional pharmacological protocol. Physicians and formulators, skilled in the art of determining doses of pharmaceutical compounds, will have no problems determining dose according to standard recommendations (Physician's Desk Reference, PDR Network (2017).

The pharmaceutical compositions described herein can be administered in a number of ways depending on whether local or systemic treatment is desired, and on the area to be treated. In one aspect, administration can be by injection, where the micronized composition is formulated into a liquid or gel. In other aspects, the micronized composition can be formulated to be applied internally to a subject. In other aspects, the micronized composition can be applied topically (including ophthalmically, vaginally, rectally, intranasally, orally, or directly to the skin).

In certain embodiments, the micronized compositions can be formulated as a topical composition applied directly to the skin. Formulations for topical administration can include, emulsions, creams, aqueous solutions, oils, ointments, pastes, gels, lotions, milks, foams, suspensions and powders. In one aspect, the topical composition can include one or more surfactants and/or emulsifiers. Surfactants (or surface-active substances) that may be present are anionic, non-ionic, cationic and/or amphoteric surfactants. Typical examples of anionic surfactants include, but are not limited to, soaps, alkylbenzenesulfonates, alkanesulfonates, olefin sulfonates, alkyl ether sulfonates, glycerol ether sulfonates, a-methyl ester sulfonates, sulfo fatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids, e.g. acyl lactylates, acyl tartrates, acyl glutamates and acyl aspartates, alkyl oligoglucoside sulfates, protein fatty acid condensates (in particular wheat-based vegetable products) and alkyl (ether) phosphates. Examples of non-ionic surfactants include, but are not limited to, fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers or mixed formals, optionally partially oxidized alk(en)yl oligoglycosides or glucoronic acid derivatives, fatty acid N-alkylglucamides, protein hydrolysates (in particular wheat-based vegetable products), polyol fatty acid esters, sugar esters, sorbitan esters, polysorbates and amine oxides. Examples of amphoteric or zwitterionic surfactants include, but are not limited to, alkylbetaines, alkylamidobetaines, aminopropionates, aminoglycinates, imidazolinium-betaines and sulfobetaines.

In certain embodiments, the surfactant can be fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, alpha-olefinsulfonates, ether carboxylic acids, alkyl oligoglucosides, fatty acid glucamides, alkylamidobetaines, amphoacetals and/or protein fatty acid condensates.

In certain embodiments, the emulsifier can be a nonionogenic surfactant selected from the following: addition products of from 2 to 30 mol of ethylene oxide and/or 0 to 5 mol of propylene oxide onto linear fatty alcohols having 8 to 22 carbon atoms, onto fatty acids having 12 to 22 carbon atoms, onto alkylphenols having 8 to 15 carbon atoms in the alkyl group, and onto alkylamines having 8 to 22 carbon atoms in the alkyl radical; alkyl and/or alkenyl oligoglycosides having 8 to 22 carbon atoms in the alk(en)yl radical and the ethoxylated analogs thereof; addition products of from 1 to 15 mol of ethylene oxide onto castor oil and/or hydrogenated castor oil; addition products of from 15 to 60 mol of ethylene oxide onto castor oil and/or hydrogenated castor oil; partial esters of glycerol and/or sorbitan with unsaturated, linear or saturated, branched fatty acids having 12 to 22 carbon atoms and/or hydroxycarboxylic acids having 3 to 18 carbon atoms, and the adducts thereof with 1 to 30 mol of ethylene oxide; partial esters of polyglycerol (average degree of self-condensation 2 to 8), trimethylolpropane, pentaerythritol, sugar alcohols (e.g. sorbitol), alkyl glucosides (e.g. methyl glucoside, butyl glucoside, lauryl glucoside), and polyglucosides (e.g. cellulose) with saturated and/or unsaturated, linear or branched fatty acids having 12 to 22 carbon atoms and/or hydroxycarboxylic acids having 3 to 18 carbon atoms, and the adducts thereof with 1 to 30 mol of ethylene oxide; mixed esters of pentaerythritol, fatty acids, citric acid and fatty alcohols and/or mixed esters of fatty acids having 6 to 22 carbon atoms, methylglucose and polyols, preferably glycerol or polyglycerol, mono-, di- and trialkyl phosphates, and mono-, di- and/or tri-PEG alkyl phosphates and salts thereof; wool wax alcohols; polysiloxane-polyalkyl-polyether copolymers and corresponding derivatives; and block copolymers, e.g. polyethylene glycol-30 dipolyhydroxystearates. In one aspect, the emulsifier is a polyalkylene glycol such as, for example, polyethylene glycol or polypropylene glycol. In another aspect, the emulsifier is polyethylene glycol having a molecular weight 100 Da to 5,000 Da, 200 Da to 2,500 Da, 300 Da to 1,000 Da, 400 Da to 750 Da, 550 Da to 650 Da, or about 600 Da.

In certain embodiments, the emulsifier is composed of one or more fatty alcohols. In one aspect, the fatty alcohol is a liner or branched C₆ to C₃₅ fatty alcohol. Examples of fatty alcohols include, but are not limited to, capryl alcohol (1-octanol), 2-ethyl hexanol, pelargonic alcohol (1-nonanol), capric alcohol (1-decanol, decyl alcohol), undecyl alcohol (1-undecanol, undecanol, hendecanol), lauryl alcohol (dodecanol, 1-dodecanol), tridecyl alcohol (1-tridecanol, tridecanol, isotridecanol), myristyl alcohol (1-tetradecanol), pentadecyl alcohol (1-pentadecanol, pentadecanol), cetyl alcohol (1-hexadecanol), palmitoleyl alcohol (cis-9-hexadecen-1-ol), heptadecyl alcohol (1-n-heptadecanol, heptadecanol), stearyl alcohol (1-octadecanol), isostearyl alcohol (16-methylheptadecan-1-ol), elaidyl alcohol (9E-octadecen-1-ol), oleyl alcohol (cis-9-octadecen-1-ol), linoleyl alcohol (9Z, 12Z-octadecadien-1-ol), elaidolinoleyl alcohol (9E, 12E-octadecadien-1-ol), linolenyl alcohol (9Z, 12Z, 15Z-octadecatrien-1-ol) elaidolinolenyl alcohol (9E, 12E, 15-E-octadecatrien-1-ol), ricinoleyl alcohol (12-hydroxy-9-octadecen-1-ol), nonadecyl alcohol (1-nonadecanol), arachidyl alcohol (1-eicosanol), heneicosyl alcohol (1-heneicosanol), behenyl alcohol (1-docosanol), erucyl alcohol (cis-13-docosen-1-ol), lignoceryl alcohol (1-tetracosanol), ceryl alcohol (1-hexacosanol), montanyl alcohol, cluytyl alcohol (1-octacosanol), myricyl alcohol, melissyl alcohol (1-triacontanol), geddyl alcohol (1-tetratriacontanol), or cetearyl alcohol.

In certain embodiments, the carrier used to produce the topical composition is a mixture polyethylene and one or more fatty alcohols. For example, the carrier is composed of 50% to 99% by weight, 75% to 99% by weight, 90% to 99% by weight, or about 95% by weight polyethylene glycol and 1% to 50% by weight, 1% to 25% by weight, 1% to 10% by weight, or about 5% by weight fatty alcohol. In a further aspect, the carrier is a mixture of polyethylene glycol and cetyl alcohol.

The topical compositions can also include additional components typically present in such compositions. In one aspect, the topical composition can include one or more of the following components: fats, waxes, pearlescent waxes, bodying agents, thickeners, superfatting agents, stabilizers, polymers, silicone compounds, lecithins, phospholipids, biogenic active ingredients, deodorants, antimicrobial agents, antiperspirants, swelling agents, insect repellents, hydrotropes, solubilizers, preservatives, perfume oils and dyes. Examples of each of these components are disclosed in U.S. Pat. No. 8,067,044, which is incorporated by reference with respect these components.

The topical compositions composed of the micronized compositions described herein can be prepared by mixing the particles with the carrier for a sufficient time such that the particles are evenly dispersed throughout the carrier. In the case when the carrier is composed of two or more components, the components can be admixed with one another prior to the addition of the micronized composition. The amount of micronized composition present in the topical composition can vary depending upon the application. In one aspect, the micronized composition is from 0.5% to 20%, 1% to 10%, 2% to 5%, or about 3% by weight of the topical composition.

### Pharmaceutical Therapeutics

In other embodiments, agents discovered to have immunomodulatory activity that enhances anti-tumor immune responses using the methods described herein are useful as a drug or as information for structural modification of existing compounds, e.g., by rational drug design. Such methods are useful for screening agents having an effect on a neoplasia.

For therapeutic uses, the compositions or agents identified using the methods disclosed herein may be administered systemically or locally to a subject to facilitate wound healing/tissue regeneration. Such agents may also be incorporated directly into a biomaterial scaffold of the disclosure to facilitate immune cell recruitment upon implantation of the scaffold. Preferable systemic routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a therapeutic wound healing agent identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplasia. Generally, amounts will be in the range of those used for other agents used in the treatment of other diseases associated with wound healing/tissue regeneration, although in certain instances lower amounts will be needed because of the increased specificity of the compound.

### Formulation of Pharmaceutical Compositions

The administration of an agent or compound or a combination of agents/compounds for the treatment of a wound may be by any suitable means that results in a concentration of the therapeutic that, combined with other components, is effective in ameliorating, reducing, or stabilizing a neoplasia. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 μg compound/kg body weight to about 5000 mg compound/kg body weight; or from about 5 mg/kg body weight to about 4000 mg/kg body weight or from about 10 mg/kg body weight to about 3000 mg/kg body weight; or from about 50 mg/kg body weight to about 2000 mg/kg body weight; or from about 100 mg/kg body weight to about 1000 mg/kg body weight; or from about 150 mg/kg body weight to about 500 mg/kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 mg/kg body weight. In other embodiments, it is envisaged that doses may be in the range of about 5 mg compound/kg body to about 20 mg compound/kg body. In other embodiments the doses may be about 8, 10, 12, 14, 16 or 18 mg/kg body weight. Of course, this dosage amount may be adjusted upward or downward, as is routinely done in such treatment protocols, depending on the results of the initial clinical trials and the needs of a particular patient.

Pharmaceutical compositions according to the disclosure may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target a neoplasia by using carriers or chemical derivatives to deliver the therapeutic agent to a particular cell type (e.g., neoplastic cell). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Methods of Treatment

In one embodiment, the present disclosure provides a method of using immunomodulatory activity to enhance anti-tumor immune responses in a subject. The methods involve administering to a subject in need thereof, an effective amount of a therapeutic combination of the disclosure. For example, a composition comprising an effective amount of an immunomodulatory agent that enhances T_{H}2 and M2 macrophage responses. Preferably, such agents are administered as part of a composition additionally comprising a pharmaceutically acceptable carrier. In a further preferable method, such agents may be applied to, or incorporated into, a biomaterial scaffold. Other embodiments include any of the methods herein wherein the subject is identified as in need of the indicated treatment.

In certain embodiments, a biocompatible scaffold and one or more immune cell modulating agents are administered to the subject. The immune cell modulating agents comprise: cytokines, monokines, chemokines, checkpoint agents, adjuvants, vaccines, antigens, chemotherapeutic agents or combinations thereof. In certain embodiments, a checkpoint agent comprises an inhibitor of programmed death-ligand 1 (PD-L1), programmed cell death protein 1 (PD-1) and/or CTLA4.

In certain embodiments, treatment includes administering to the subject CD4⁺ T cells, wherein the CD4⁺ T cells are autologous, haploidentical, or combinations thereof.

In certain embodiments, stem cells, chimeric antigen T (CAR-T) cells, CAR natural killer cells (CAR-NK), bone marrow cells or combinations thereof, are administered to the subject.

The cells may be induced progenitor cells. The cells may be cells isolated from a subject, e.g., a donor subject, which have been transfected with a stem cell associated gene to induce pluripotency in the cells. The cells may be cells which have been isolated from a subject, transfected with a stem cell associated gene to induce pluripotency, and differentiated along a predetermined cell lineage. The cells may be cells including a vector expressing a desired product. These or any other types of cells may be used for transplantation or administration to a subject in need of therapy.

In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4⁺ and/or of CD8⁺ T cells are naive T (T_{N}) cells, effector T cells (T_{EFF}), memory T cells and sub-types thereof, such as stem cell memory T (T_{SCMX} central memory T (T_{CM} effector memory T (T_{EM}), or terminally differentiated effector memory T cells, tumor-infiltrating lymphocytes (T_{IL}), immature T cells, mature T cells, helper T cells, cytotoxic T cells, mucosa-associated invariant T (MAIT) cells, naturally occurring and adaptive regulatory T (Treg) cells, helper T cells, such as T_{H}1 cells, T_{H}2 cells, T_{H}3 cells, T_{H}17 cells, T_{H}9 cells, T_{H}22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

### Combination Therapies

Compositions of the invention may be combined in a pharmaceutical combination formulation, or dosing regimen as combination therapy, with a second compound, for example, chemotherapeutic agents, agents used in the treatment of autoimmune diseases, etc. The second compound of the pharmaceutical combination formulation or dosing regimen preferably has complementary activities to the compounds of the invention such that they do not adversely affect the other(s). Such molecules are suitably present in combination in amounts that are effective for the purpose intended.

The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein preferably there is a time period while both (or all) active agents simultaneously exert their biological activities. Suitable dosages for any of the above coadministered agents are those presently used and may be lowered due to the combined action (synergy) of the newly identified agent and other chemotherapeutic agents or treatments.

The combination therapy may provide “synergy” and prove “synergistic”, e.g. the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g. by different injections in separate syringes. In general, during alternation therapy, an effective dosage of each active ingredient is administered sequentially, e.g. serially, whereas in combination therapy, effective dosages of two or more active ingredients are administered together.

As an example, the agent may be administered in combination with surgery to remove an abnormal proliferative cell mass. As used herein, “in combination with surgery” means that the agent may be administered prior to, during or after the surgical procedure. Surgical methods for treating epithelial tumor conditions include intra-abdominal surgeries such as right or left hemicolectomy, sigmoid, subtotal or total colectomy and gastrectomy, radical or partial mastectomy, prostatectomy and hysterectomy. In these embodiments, the agent may be administered either by continuous infusion or in a single bolus. Administration during or immediately after surgery may include a lavage, soak, or perfusion of the tumor excision site with a pharmaceutical preparation of the agent in a pharmaceutically acceptable carrier. In some embodiments, the agent is administered at the time of surgery as well as following surgery in order to inhibit the formation and development of metastatic lesions. The administration of the agent may continue for several hours, several days, several weeks, or in some instances, several months following a surgical procedure to remove a tumor mass.

The subjects can also be administered the agent in combination with non-surgical anti-proliferative (e.g., anti-cancer) drug therapy. In one embodiment, the agent may be administered with a vaccine (e.g., anti-cancer vaccine) therapy. In one embodiment, the agent may be administered in combination with an anti-cancer compound such as a cytostatic compound. A cytostatic compound is a compound (e.g., a nucleic acid, a protein) that suppresses cell growth and/or proliferation. In some embodiments, the cytostatic compound is directed towards the malignant cells of a tumor. In yet other embodiments, the cytostatic compound is one that inhibits the growth and/or proliferation of vascular smooth muscle cells or fibroblasts.

Suitable anti-proliferative drugs or cytostatic compounds to be used in combination with the agents of the invention include anti-cancer drugs. Anti-cancer drugs are well known and include: Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-Ia; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Taxotere; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinflunine; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

According to the methods of the invention, the agents of the invention may be administered prior to, concurrent with, or following the other therapeutic compounds or therapies. The administration schedule may involve administering the different agents in an alternating fashion. In other embodiments, the agent may be delivered before and during, or during and after, or before and after treatment with other therapies. In some cases, the agent is administered more than 24 hours before the administration of the second agent treatment. In other embodiments, more than one anti-proliferative therapy or an autoimmune therapy may be administered to a subject. For example, the subject may receive the agents of the invention, in combination with both surgery and at least one other anti-proliferative compound. Alternatively, the agent may be administered in combination with more than one anti-cancer drug.

### Kits or Pharmaceutical Systems

The present compositions may be assembled into kits or pharmaceutical systems for use in induce an anti-tumor immune response. Kits or pharmaceutical systems according to this aspect of the disclosure comprise a carrier means, such as a box, carton, tube or the like, having in close confinement therein one or more container means, such as vials, tubes, ampoules, bottles and the like. The kits or pharmaceutical systems of the disclosure may also comprise associated instructions for using the agents of the disclosure. Kits of the disclosure include at least one or more immunomodulators. If desired, the kit also includes a reagent to be used as a biomaterial scaffold. The kit may include instructions for administering the immunomodulatory agent in combination with one or more agents, such as chemotherapeutic agents.

The invention has been described in detail with reference to preferred embodiments thereof. However, it will be appreciated that those skilled in the art, upon consideration of this disclosure, may make modifications and improvements within the spirit and scope of the invention.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

### Example 1: Acellular Biologic Scaffolds Generate an Anti-Tumoral Immune Microenvironment

Wound healing and tumor development share similarities that present the question of whether regenerative medicine therapies can create a pro-tumorigenic environment. Biologic scaffold materials derived from decellularized tissues produce a highly pro-regenerative environment through immune-mediated mechanisms.

In order to address concerns about potential pro-carcinogenic effects of ECM, the present study investigated the in vivo effect of an FDA approved pro-regenerative urinary bladder ECM material (UBM) on the establishment of the tumor microenvironment primarily using the B16-F10 melanoma model. The results obtained as described herein showed that UBM particle scaffolds inhibited tumor formation in an immune dependent manner, increasing both myeloid and lymphoid cell recruitment and promoting a unique T_{H}2-M2 type 2 immune profile. The in depth immune analysis conducted herein showed that while there is overlap between the regenerative and tumorigenic immune signatures that possess a Type-2 bias, they are distinct phenotypes. The pro-regenerative immune phenotype does not promote tumor development or metastasis. The immune microenvironment induced by biological scaffolds can however be potentiated by anti-PD1 or anti-PDL1 checkpoint blockade, enhancing tumor growth suppression.

Materials and Methods

Injectable biomaterials and reagents. Lyophilized urinary bladder matrix (UBM) particulate was obtained from ACell Inc. UBM was produced in a facility adhering to good manufacturing practices (GMP) and terminally sterilized for clinical or pre-clinical application. The proteomic composition of UBM has been described previously (12). Synthetic particulate material controls include Alum (endotoxin free 2% aluminum hydroxide gel, Alhydrogel, InvivoGen) and mesoporous silica SBA-15 (<150 μm particle size, pore size 8 nm, Sigma-Aldrich). IL4 complex (IL4c) was prepared by mixing 20 μg of recombinant murine IL4 (PeproTech) to 100 μg anti-IL4 antibody (clone 11B11, BioXcell) for a 1:5 weight ratio of IL4:anti-IL4 (equivalent to a 1:2 molar ratio) for 20 minutes on ice. IL4c was incubated with UBM at least 30 min before injection.

Mice. Wild type C57BL/6 and balb/c mice were obtained from Charles River Laboratories or The Jackson Laboratories. Lymphocyte deficient Rag1^{-/-} (B6.129S7-Rag1tm1Mom/J) were obtained from Jackson Laboratories. Each experiment used mice matched from the same facility.

Cell culture. B16-F10 (CRL-6475) melanoma and CT26 (CRL-2638) colorectal carcinoma tumor cell lines were obtained from the American Type Cell Culture Collection (ATCCC). Luciferase transduced B16-F10 cells were obtained from Perkin Elmer, Inc. All cell lines were grown in DMEM media supplemented with 10% heat-inactivated FCS (Hyclone), 2 mM L-glutamine, 100 U/ml penicillin G, and 100 μg/ml streptomycin (Hyclone).

In vitro UBM biocompatibility. The effect of UBM particles on B16-F10 melanoma adhesion, viability, and growth was determined in vitro. Coverslips were coated with 0.4 mg/cm² UBM or Type I collagen (Sigma-Aldrich) as previously described (32). B16-F10 melanoma cells were seeded in triplicate on coated and uncoated 12 mm coverslips at a density of 15,000 cells/cm² and given 1.5 hours to attach. Non-adherent cells were then removed with 3 washes with PBS and the remaining cells incubated with Calcein-AM viability dye (Thermo Fisher) for 20 min. Coverslips were then imaged for viable cell adhesion and quantified by counting.

Scanning electron microscopy (SEM). The topography of UBM particles before and after implantation was characterized by SEM. Post-implantation UBM was carefully dissected from mice after 3 days and fixed in 2.5% glutaraldehyde, 3 mM MgCl, 0.1M cacodylate buffer (pH 7.2) for 24 hours at 4° C. with agitation. Samples were rinsed three times with cacodylate buffer and further fixed with 1% osmium tetroxide in cacodylate buffer for 1 hour at room temperature. Samples were rinsed with water and dehydrated with a graded series of ethanol (30%, 50%, 70%, 90%, and three times in anhydrous 100% ethanol) for 15 min with agitation during each step. Samples were dried with a 1:1 solution of hexamethyldisilizane (HMDS):100% ethanol and two additional changes of 100% HMDS for 15 min each followed by overnight dessication. Dried implants and pre-implant particles were sputter coated with 10 nm gold/palladium alloy and imaged using a LEO (Zeiss) field-emission SEM with 1 k accelerating voltage.

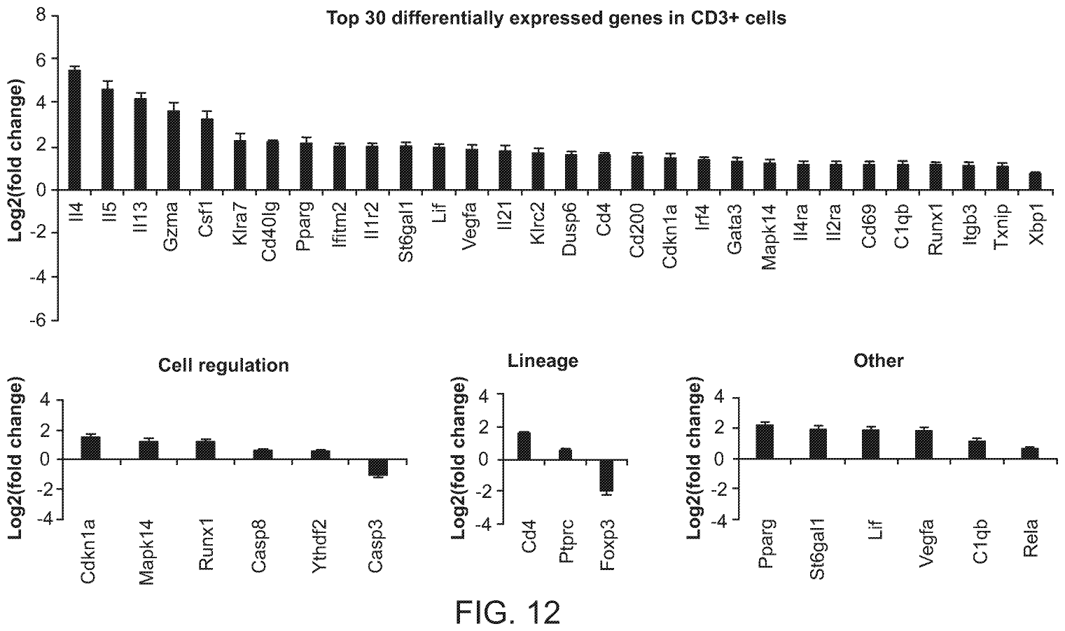

Clodronate liposome macrophage depletion. Circulating macrophage progenitors were partially ablated by systemic administration of clodronate liposomes (5 mg/ml, clodronateliposomes.com) to determine the role of macrophages in the UBM and tumor immune response. Clodronate loaded liposomes or PBS loaded controls (1 mg liposome/20 g mouse) were injected intraperitoneally four and two days prior to cancer cell implantation and on the day of implantation to clear macrophages. The depletion was maintained every other day thereafter until sacrifice as described in FIG. 12. To verify efficacy of depletion, peripheral blood was collected into EDTA solution and red blood cell lysis performed (Ammonium-Chloride-Potassium lysis buffer) for myeloid cell marker staining and flow cytometry.

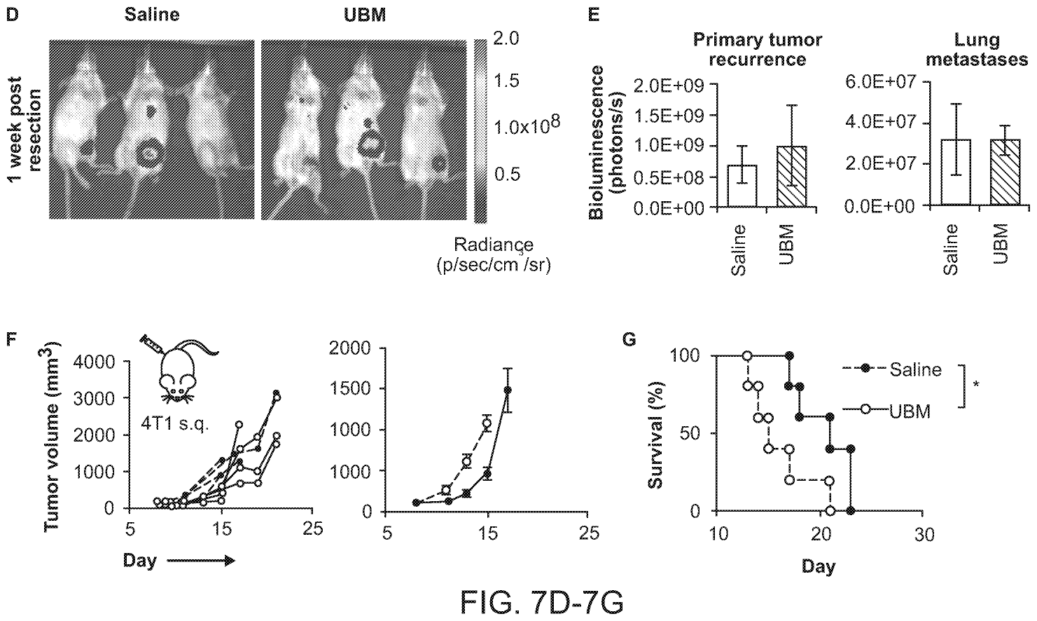

Subcutaneous tumor formation. The syngeneic cancer lines B16-F10 melanoma, CT26 colorectal carcinoma, and 4T1 mammary carcinoma were implanted subcutaneously in 7-8 week old female C57BL/6 or balb/c mice, with and without UBM. Cells were used within the same two passages for all experiments. UBM particles were hydrated with phosphate buffered saline and thoroughly mixed with cell suspension to a final concentration of 50 mg UBM (dry wt)/ml. The right flanks of mice were shaved, disinfected with 70% ethanol, and injected with 1×10⁵ cancer cells suspended in 100 μl of saline or UBM (5 mg of UBM particles per injection). This amount was determined from a dose response study using 12.5, 25, and 50 mg UBM/ml suspension (FIGS. 7A-7G). Tumor dimensions were monitored by external measurements using digital calipers. Tumor volume was calculated by the following equation where L is the tumor length (larger dimension) and W is the width (smaller dimension):

$$Tumor volume = \frac{π}{6} ( L \times {W}^{2} )$$

Mice were sacrificed by carbon dioxide asphyxiation once tumors grew to 19.5-20 mm in any dimension according to Johns Hopkins Animal Care and Use Committee policy. Survival was defined as the number of days to sacrifice.

Orthotopic breast cancer tumor formation and resection model. The 4T1 mammary carcinoma line (expressing firefly luciferase) was used to evaluate the effect of UBM implantation on tumor recurrence at the primary location and lung metastasis after resection in 8 week old female balb/c mice. Mice were anesthetized and the surgical site shaved/disinfected. A 1 cm incision was made over the right flank for injection of 1×10⁶ 4 T1 cells suspended in 50 μl of MatriGel (BD Biosciences) directly into the right abdominal mammary fat pad. The incision was closed with single interrupted Vicryl suture and the animals allowed to ambulate normally. Once 4T1 tumors grew to approximately 1 cm in greatest dimension (day 10), a second surgery performed to remove the tumor bulk. Following removal of the entire visible tumor mass, 0.2 ml of a 100 mg/ml UBM particle suspension was injected into the space compared to Saline. Tumor volume at the primary resection site was monitored by external measurement and lung metastasis by live animal bioluminescence imaging.

In vivo bioluminescence imaging of tumors. Early melanoma tumor growth and 4T1 breast cancer recurrence/metastasis in the presence of UBM biomaterial was characterized by live animal bioluminescence imaging with the IVIS Spectrum In Vivo Imaging System (Perkin Elmer). Firefly luciferase expressing cancer cells (B16-F10 or 4T1) were injected with saline or UBM particle suspension as described above for non-invasive imaging. B16-F10 melanoma was imaged after 1, 3, and 5 days post implantation with UBM, and 4T1 breast cancer tumors imaged 1 week after primary tumor resection and implantation with UBM. Each mouse was intraperitoneally injected with 150 mg/kg of D-Luciferin/K⁺ (XenoLight, Perkin Elmer) 15 minutes prior to imaging, and then anesthetized via isofluorane inhalation and imaged with a range of exposure times. The injection site (right flank region) of each mouse was analyzed for normalized luminescent flux (photons/s) to determine tumor growth. Additionally, the chest region of mice used in the 4T1 resection model were imaged to quantify lung metastases. WT B16-F10 cells delivered with saline or UBM (N=2 each treatment group) were used as negative controls to confirm specificity of the bioluminescent signal.

Checkpoint blockade immunotherapy following UBM implantation. B16-F10 cells were delivered subcutaneously into the right flank of 7-8 week old female C57BL/6 mice with Saline or UBM as described. Eight days following implantation, monoclonal antibodies blocking either PD-1 (clone RMP1-14, InVivoPlus grade, BioXCell), PD-L1 (clone 10F.9G2, InVivoPlus grade, BioXCell), or PD-L1 (clone TY25, InVivoMab grade, BioXCell) were delivered intraperitoneally at 5 mg/kg body weight. Checkpoint blocking antibodies were delivered every 3 days for a total of 4 treatment doses. Tumor volume and survival were monitored. (N=8-10 for each treatment group). IgG2a (clone 2A3, InVivoPlus grade, BioXCell) and IgG2b (clone LTF-2, InVivoPlus grade, BioXCell) isotype controls were delivered using the same schedule (N=5 each isotype which were then pooled for analysis).

The therapeutic effect of UBM treatment on the ability of B16-F10 melanoma cells to form tumors with checkpoint immunotherapy was tested by delayed UBM injection. B16-F10 cells were injected subcutaneously into the right flank of 7-8 week old female C57BL/6 mice and the skin over the injection site labeled with a permanent marker. One day later, after cells had engrafted in the subcutaneous space, 200 μl of UBM particles (50 mg/ml) or Saline was injected in the same approximate area. Four days after UBM or Saline injection (day 5 after B16-F10 implantation), anti-PD-1 monoclonal antibody or isotype control was delivered following the same dosing schedule as above (5 mg/kg body weight, 4 injections spaced 3 days apart). Tumor volume and survival were monitored (N=5 per group)

B16-F10 Tumor histology and immunolabeling. Histologic analysis of tumors was conducted after 7 days of growth and after tumors had grown to a volume of 200 mm³. Whole tumors and UBM were explanted, fixed for 2 days in neutral buffered formalin, and dehydrated with a graded series of ethanol: 70%, 80%, 95% (2×) and 100% (3×) for at least 1 hour each. Tumors were cleared with 3× 1 hour changes of Xylene and then infiltrated with several changes of paraffin wax. Embedded tumors were cut into 5 μm sections for H&E staining or immunofluorescent (IF) staining. IF staining was conducted for tumor infiltrating T cells (CD3) and B cells (B220). Sections were deparaffinized and underwent antigen retrieval in citrate buffer (0.01 M citrate, pH 6) for 20 minutes in a vegetable steamer. Nonspecific binding was blocked for 1 hour and then incubated with primary antibodies overnight at 4° C.: rat anti-B220/CD45R (clone RA3-6B,2 biolegend) and rabbit anti-CD3 (clone SP7, abcam) monoclonal antibodies. Following washing, secondary antibodies were added for 1 hour at room temperature: goat anti-rat Alexa Fluor-488 (Thermo Fisher) and goat anti-rabbit Alexa Fluor-568. Background fluorescence was quenched by incubating in Sudan Black B (Sigma) in 70% ethanol for 20 min. Sections were rinsed with water, counterstained with DAPI, coverslipped, and imaged. Spleen tissue was processed and stained as described for tumors except using the CD3 primary antibody in combination with either rat anti-CD4 (clone 4SM95, Thermo Fisher) or rat anti-CD8a (clone 4SM16, Thermo Fisher) monoclonal antibodies.

CD4 T cell adoptive transfer in Rag1^{-/-} mice. The role of CD4⁺ T cells in the UBM response was assessed by repopulating lymphocyte Rag1^{-/-} mice with purified CD4⁺ cells. Pooled spleens and lymph nodes from 5 week old female WT C57BL/6 mice were harvested and prepared as a single cell suspension using the gentleMACS automated tissue dissociator (Miltenyi). Tissue was digested with the supplied enzyme mix using the manufacturer's programmed spleen dissociation cycle for 15 min at 37° C. CD4 cells were isolated by negative selection using magnetic assisted cell sorting (MACS, Miltenyi) following the manufacturer's instructions. Cells were incubated with biotinylated antibodies against CD8a, CD11b, CD11c, CD19, CD45R (B220), CD49b, CD105, MHC Class II, Ter-119, and TCRγ/δ, and reactive cells removed by binding to magnetic beads. The purity of the cell suspension was verified with flow cytometry. Purified CD4⁺ T cells were transferred to 5 week old Rag1^{-/-} mice by tail vein injection (4 million viable cells per mouse). Repopulation was verified 12 days later by flow cytometry. Peripheral blood was collected into EDTA solution, red blood cell lysis performed, and stained for lymphocyte markers. Age matched CD4 repopulated Rag1^{-/-} mice, Rag1^{-/-} controls, and WT mice were challenged with B16-F10 cells with and without UBM 17 days after repopulation.

Flow cytometry and cell sorting from tumors and lymphoid tissues. Tumor/UBM immune cell infiltrates were characterized by flow cytometry using the antibodies and fluorescent dyes presented in Table 1. All flow cytometry data was collected using a BD LSR II flow cytometer or sorted using a BD FACSAria II, and data were analyzed using FlowJo software (Tree Star).

For analysis of tumor immune populations, tumors/UBM were explanted into RPMI media on ice and finely minced and digested with 0.5 mg/ml Liberase TL (Roche) and 0.2 mg/ml DNAse I (Roche) for 45 minutes at 37° C. with agitation. The suspension was then passed through a 100 μm cell strainer and washed. Small tumors at the 7 day time point were passed through a 70 μm cell strainer and proceeded directly to staining. Larger tumors at the 14 day time point or later underwent density separation using a Percoll gradient (GE Healthcare Life Sciences) to remove excess necrotic cells and debris. One part 10×PBS was added to 9 parts Percoll (1.13 g/ml) to create a 100% Percoll solution, which was then diluted to 80%, 40%, and 20% Percoll solutions with PBS. The filtered cell suspension was centrifuged and suspended in 4 ml 80% Percoll, which was subsequently layered with 4 ml of the 40% and then 3 ml of the 20% solutions above it. Tubes were centrifuged at 1,000 g for 20 min at room temperature and the resulting interfacial layer between the 80% and 40% layers collected for staining. Lymphoid tissues (lymph node and spleen) were harvested, diced, and digested with 0.25 mg/ml Liberase TL (Roche) and 0.2 mg/ml DNAse I (Roche) for 25 minutes at 37° C. with agitation. Lymphoid suspensions were filtered through a 100 μm cell strainer, washed, and proceeded directly to cell staining.

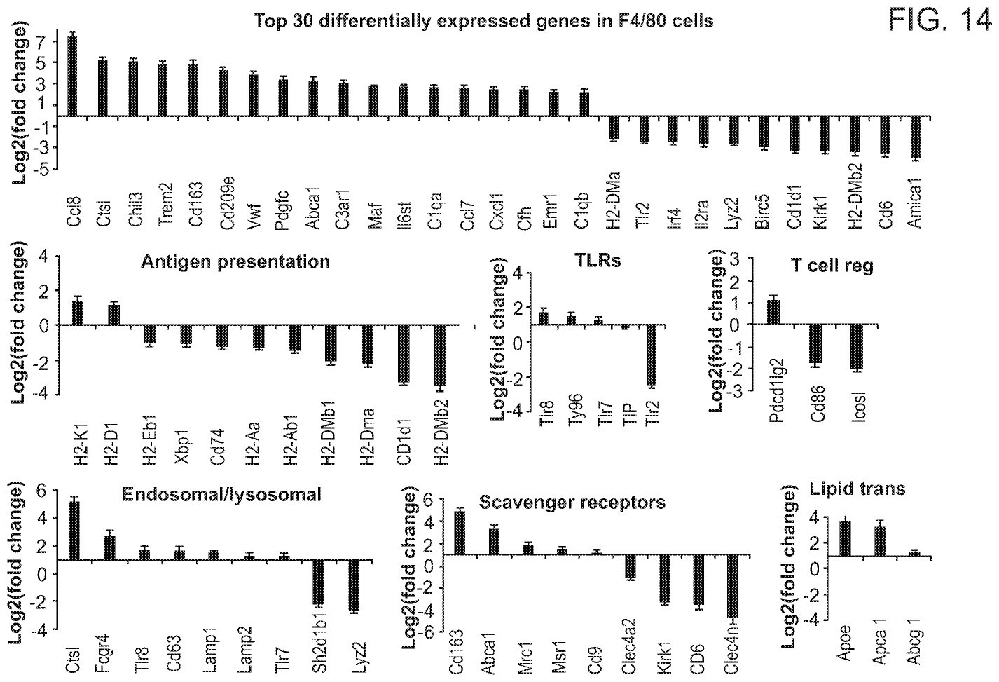

Surface staining for flow cytometry was conducted in round bottom 96-well plates on ice and in the dark. Viability staining was conducted for 20 min, followed by a surface staining cocktail for 45 min on ice with non-specific binding blocked by anti-CD16/32. The myeloid staining panel consisted of Viabilitiy-eFluor780, CD45-BV605, CD11b-AF700, MHCII-AF488, Siglec F-PE/CF594, Ly6C-PerCP/Cy5.5, Ly6G-Pacific Blue, F4/80-PE/Cy7, CD11c-APC, CD206-PE, CD86-BV510. T cell surface staining consisted of Viability-Aqua, CD45-PerCP/Cy5.5, CD19-PE, CD3-AF488, NK1.1-APC, CD4-PE/Cy7, CD8-AF700, CD62L-APC/Cy7, CD44-BV605. Full antibody information is listed in Table 1. Cell sorting was conducted immediately, whereas analysis samples were fixed with Cytofix/Cytoperm (BD) for 25 min on ice. Small 7 day saline tumors were pooled prior to sorting to increase immune cell yield. All tumor derived samples were stored in PBS buffer with 2 mM EDTA and passed through a 40 μm cell strainer before analysis/sorting. CD3⁺ and F4/80⁺ cell populations were sorted (FIG. 14) for NanoString analysis with the following panel: Viability-eFluor780, CD45-BV605, CD11b-AF700, CD3-APC, F4/80-PE/Cy7.

Cells isolated for intracellular cytokine experiments were stimulated for 5 hours at 37° C. in a cell stimulation cocktail with transport inhibitors (eBioscience) consisting of phorbol 12-myristate 13-acetate (PMA), ionomycin, brefeldin A, and monensin in RPMI media supplemented with 10% FBS, 2 mM glutamine, 1% non-essential amino acids (Gibco), 20 mM HEPEs buffer, 1 mM sodium pyruvate, and 55 μM 2-mercaptoethanol. Cells were then placed on ice, washed, and stained with Viability-Aqua followed by surface staining: CD45-PerCP/Cy5.5, CD3-AF488, CD4-PE/Cy7, CD8-AF700. Cells were fixed and permeabilized for 20 min on ice with Cytofix/Cytoperm (BD Biosciences) and washed with Perm/Wash buffer (BD Biosciences). Intracellular staining was then conducted using antibodies diluted in Perm/Wash buffer for 45 min on ice, followed by washing in Perm/Wash buffer. The following intracellular staining antibodies were used: FoxP3-Pacific Blue, IL4-PE, IFNy-BV605.

Nanostring gene expression analysis. Sorted tumor infiltrating F4/80⁺ and CD3⁺ cell were analyzed for gene expression using the Nanostring Pan Cancer Immune Profiling Panel (XT-CSO-MIP1-12, NanoString Technologies, Inc.). Cells were directly sorted into RLT lysis buffer with 2-mercaptoethanol for RNA purification using the RNEasy micro kit (Qiagen) following the manufacturer's instruction. RNA concentrations were determined using the Qubit RNA HS Assay Kit (Thermo Fisher). For F4/80+ cells, 25 μg of RNA was added to capture and barcoded detection probes, and hybridized for 18 hours at 65° C. CD3+ cells underwent 5 rounds of pre-amplification with the Low RNA Input Kit (PP-MIP1-12, Nanostring) followed by a 20 hour hybridization at 65° C. All hybridized samples were purified using a NanoString Prep Station operating under high sensitivity mode and mRNA transcripts counted using the nCounter digital analyzer system (Nanostring). Data was analyzed using nSolver software (v3.0, NanoString). Gene expression for each sample was normalized to the geometric mean of the reference genes: Oaz1, Hprt, Polr2a, Sdha, Hdac3, and Alas1 for F4/80⁺ cells, and Oaz1, Hprt, Polr2a, Sdha, Hdac3, Rpl19, Ppia, G6pdx, and Sf3a3 for CD3⁺ cells. Reference genes were selected for stability across conditions.

Statistical analysis. All tumor volume, survival, and flow cytometry statistical analysis was conducted using Prism software (GraphPad Software, Inc.) with significance defined as P<0.05. All survival data was analyzed with the log-rank test compared to WT saline with the Sidak correction for multiple comparisons. All tumor volume growth curves were analyzed by two-way repeated measures ANOVA with post-hoc Tukey test at each time point before sacrifice. All flow cytometry data was analyzed with a student's t-test compared to a control group as indicated in figure legends. Nanostring differential expression was analyzed using nSolver software (v3.0, Nanostring). Only genes with a minimum of 20 counts (equivalent to three standard deviations over average background) in greater than 50% of samples were analyzed. False discovery rate adjusted p-values were determined for each gene using the Benjamini-Yekutieli method.

### Results

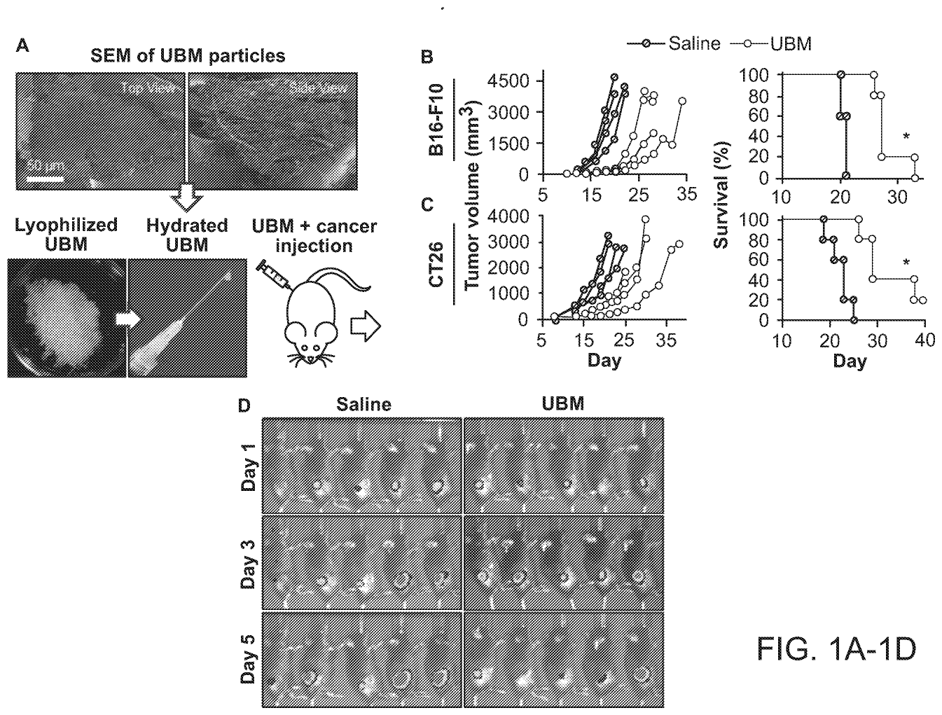

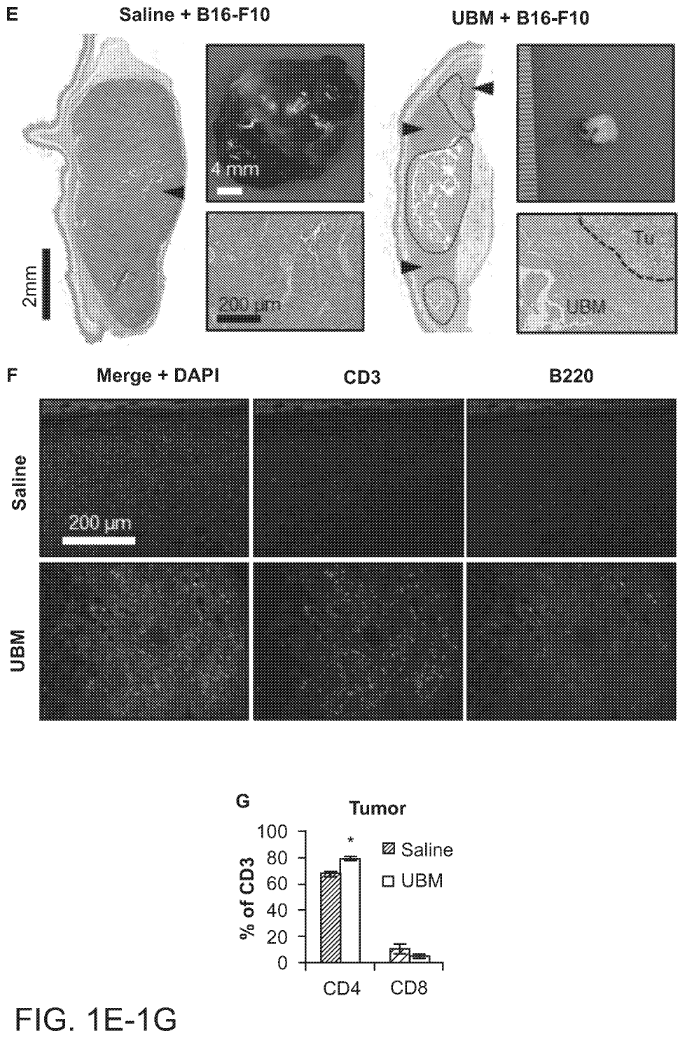

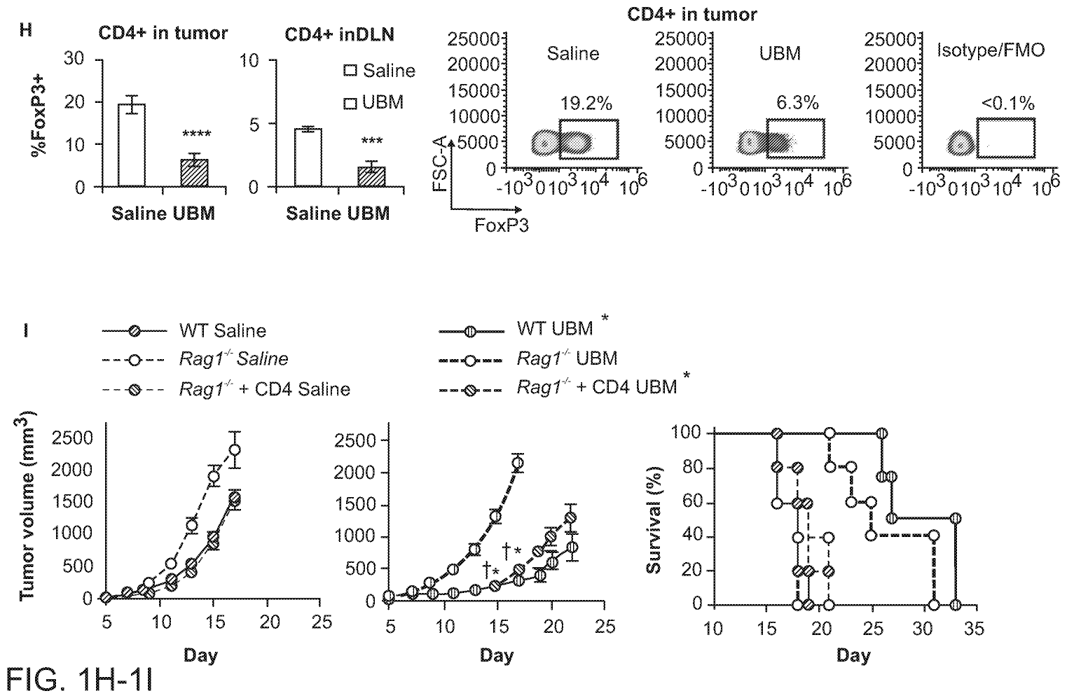

UBM ECM consists of the decellularized basement membrane and tunica propria layers of porcine urinary bladder with a previously defined proteomic composition and structure(12, 13). UBM can be comminuted into small particles that retain a lamellar sheet architecture with sizes ranging between 20-150 μm in greatest dimension as shown by scanning electron microscopy (FIG. 1A). UBM particles can be hydrated and suspended in saline solution as an injectable formulation, which is advantageous for repair of irregular three dimensional tissue defects. Though ECM scaffolds have been prepared from numerous mammalian (allogeneic and xenogeneic) sources (6), similarly processed ECM materials apparently elicit comparable functional repair outcomes in many instances (7). UBM is representative of an FDA cleared, clinically utilized ECM scaffold material.

Since ECM materials have been shown to elicit a unique, pro-healing microenvironment, it was sought to determine how this response affects tumor formation. The effect of UBM was first evaluated on cancer growth in an orthotopic breast cancer and reconstruction model. 4T1 breast cancer tumors were induced in the mammary fat pad of female balb/c mice, the bulk tumor resected, and UBM particles or a Saline control implanted into the resulting defect (FIG. 7A). Consistent with previous reports, the UBM did not enhance growth at the primary tumor site or lung metastases (FIGS. 7B-7E). Furthermore, it was observed that primary tumor regrowth occurred within nearby tissues that 4T1 cells had invaded such as the dermis or body wall, and not from cells in close proximity with UBM. Therefore the models were switched to allow characterization of the direct effect of UBM scaffold immune responses on tumor formation using a modified subcutaneous tumor model system. Cancer cell lines were mixed with 50 mg/ml UBM particles or saline, and injected subcutaneously into the flanks of syngeneic mouse strains. This model permits evaluation of the direct interaction between the implantable ECM microenvironment and initial tumor formation.

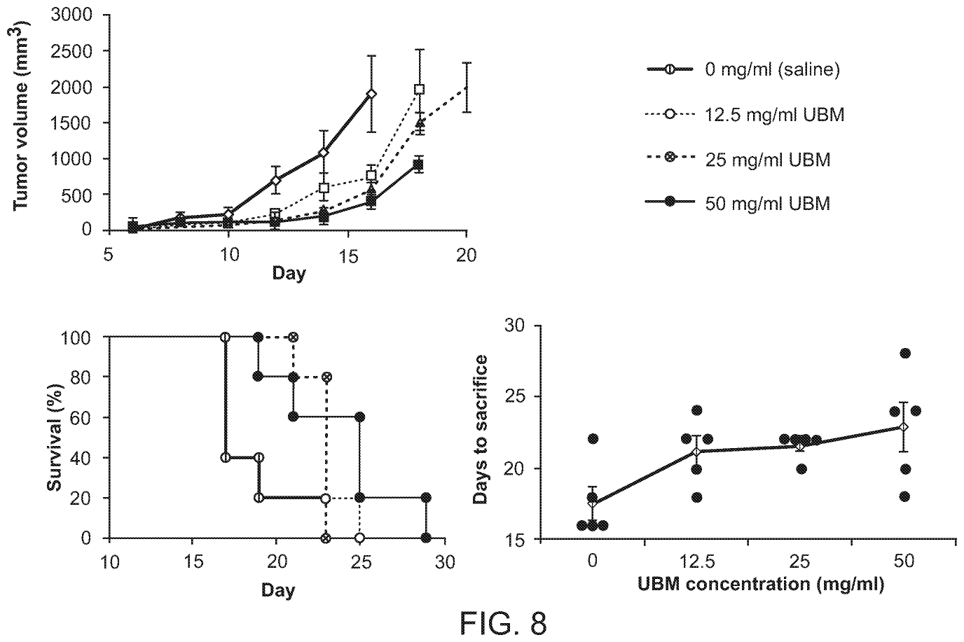

UBM particles consistently delayed tumor formation when co-implanted with syngeneic cancer cell lines: 4T1 breast cancer (FIG. 7F, 7G), B16-F10 melanoma (FIG. 1B) and CT26 colon carcinoma (FIG. 1C) were all delayed to various extents indicating that tumor growth inhibition was not cell line or strain specific. This delay resulted in increased survival times by approximately 6 days in the B16-F10 model (21.2±0.5 days for Saline vs 28.8±1.3 days for UBM) and by 10 days in the CT26 model (22.2±1.0 days for Saline vs 32.4±2.8 days for UBM) (FIGS. 1B, 1C). Before tumors were palpable (by 7 days) live animal bioluminescent imaging of Luciferase expressing B16-F10 cells confirmed live tumor cell engraftment with the UBM implant after 1 day, but suggest growth is slowed within the first 1-5 days of implantation (FIG. 1D). The focus then was on the B16-F10 melanoma model for subsequent experiments because this is a fast growing and relatively poorly immunogenic tumor with little lymphocyte infiltration. Using the B16-F10 model, it was confirmed that this UBM concentration (50 mg/ml) was optimal as it maximized tumor inhibition while also remaining easily injectable (FIG. 8).

Histologic analysis was conducted at different time points for Saline (day 10) and UBM delivery (16 days) in order to compare groups at a similar external tumor volume. Saline delivered tumors display the typical B16-F10 melanoma morphology; a high density of melanocytes, necrotic regions, and large but poorly developed blood vessels (FIG. 1E). In contrast, tumor formation begins as small nodules (FIG. 1E, arrowheads) among UBM particles (FIG. 1E, dashed line), with a dense immune cell infiltrate at the host-UBM interface. Histology also affirms that the UBM material itself is present for at least 2 weeks during initial tumor growth, indicating that external tumor volume measurements are an overestimation of tumor size that includes the volume occupied by the UBM implant itself.

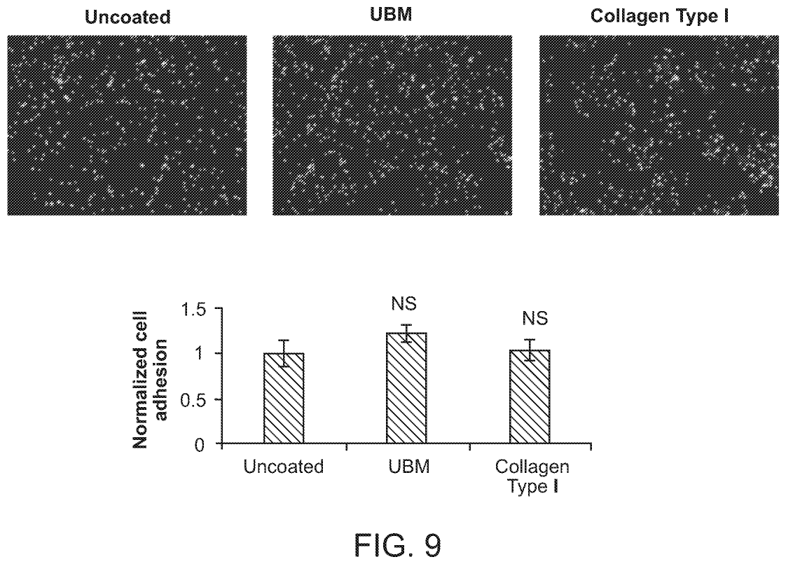

CD4⁺ T cells are required for UBM mediated tumor growth inhibition. Potential factors were then investigated that may explain delayed tumor formation when B16-F10 cells were implanted with UBM materials. Cytocompatibility was excluded as a direct cause of inhibition since neither B16-F10 viability nor adhesion was affected by UBM in vitro (FIG. 9), consistent with bioluminescence imaging that showed live cell engraftment after 1 day in vivo (FIG. 1D). Since UBM has been shown to be strongly immunomodulatory when used in wound repair applications, it was sought to elucidate whether the immune response to UBM was influencing B16-F10 tumor growth. Adaptive immunity is a vital component of cancer immune surveillance, and indeed it was found that a greater density of CD3⁺ T cells recruited to the host-UBM interface after 7 days compared to B16-F10 tumors delivered with Saline, which were largely non-immunogenic (FIG. 1F). B220⁺ B cells were also more numerous in response to UBM than saline delivered tumors, although to a lesser extent than T cells.

Flow cytometry analysis of tumor infiltrating lymphocytes at 7 days post implantation (FIG. 1G) showed that a greater proportion of UBM recruited T cells were CD4⁺ (79.14±3.5%) compared to Saline (67.6±1.0%) Immunosuppressive regulatory T cells (Tregs) aid in cancer immune escape and also express CD4, however a decrease in Tregs with UBM implantation was found instead. The proportion of CD4⁺FoxP3⁺Tregs decreased from 19.22% to 6.33% with UBM implantation compared to saline, and from 4.54% to 1.64% in the tumor draining lymph node (DLN) after 7 days (FIG. 1H). This provides evidence that UBM recruited T cells are primarily CD4⁺ T helper (Th) cells.

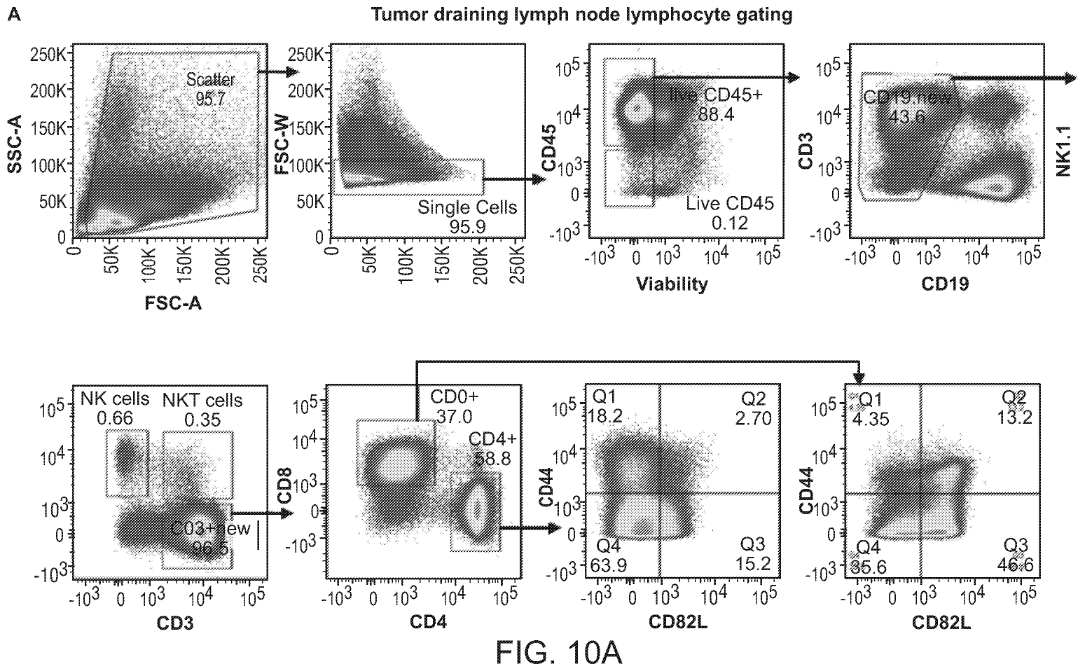

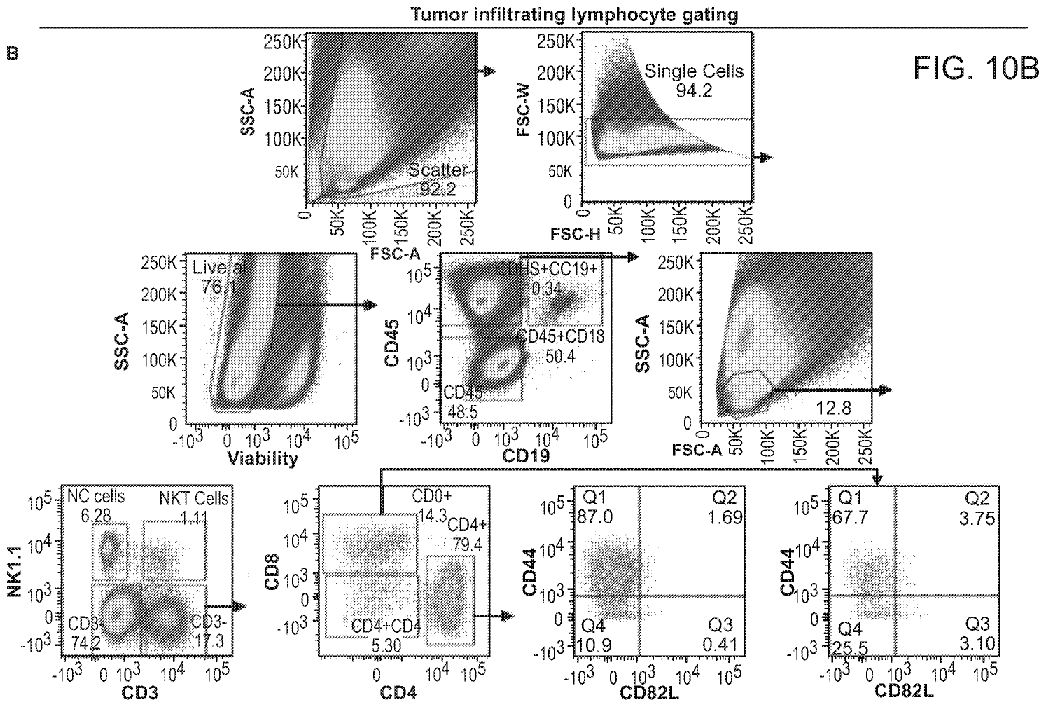

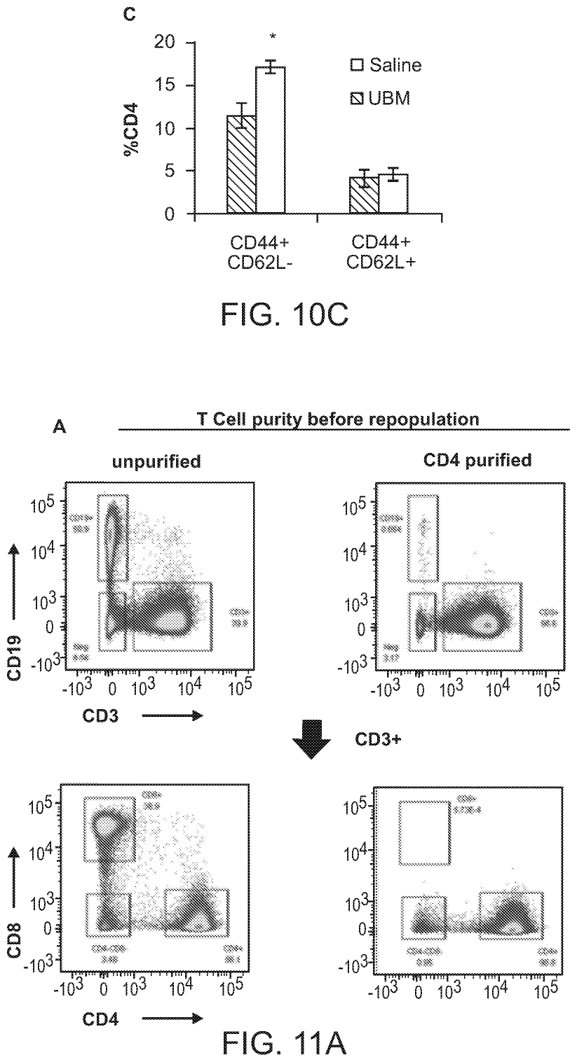

Given the observed inhibitory effect of the UBM microenvironment on tumor growth concomitant with altered lymphocyte recruitment, this model was tested in Rag1^{-/-} mice which lack mature T and B cells. It was found that in the absence of mature lymphocytes, the tumor inhibitory effect of UBM delivery was completely ablated (FIG. 1I). Tumors grew rapidly with UBM delivery in Rag1^{-/-} mice with no difference from Saline delivery. Since it was found that CD4⁺ T cells were preferentially recruited to ECM and previous studies had demonstrated that CD4⁺ T helper cells were crucial to the tissue healing response facilitated by ECM implantation (4), it was investigated whether CD4⁺ T helper cells were also responsible for tumor inhibition in UBM scaffolds. Purified CD4⁺ T cells were intravenously transferred into Rag1^{-/-} mice 17 days prior to B16-F10 and UBM implantation to populate the T helper cell compartment (>99% purity), which was maintained to study endpoint (FIGS. 10A-10C). The addition of CD4⁺ cells Rag1^{-/-} largely rescued UBM induced B16-F10 tumor growth inhibition observed in WT animals. Tumor growth with Saline delivery in CD4⁺ cell populated Rag1^{-/-} mice was indistinguishable from WT. Notably, early tumor inhibition kinetics with UBM was recapitulated with the addition of CD4⁺ T cells (FIG. 21) and corresponded to improved survival following delivery with UBM from 18.6±0.4 days in Rag1^{-/-} mice to 26±2.1 days with CD4⁺ T cell repopulation. The Rag1^{-/-} and CD4⁺ repopulation studies confirm that the retarded tumor growth with the UBM is not due to any impact on tumor cell viability or engraftment efficiency caused by the scaffold itself, but rather is an immune related phenomenon.

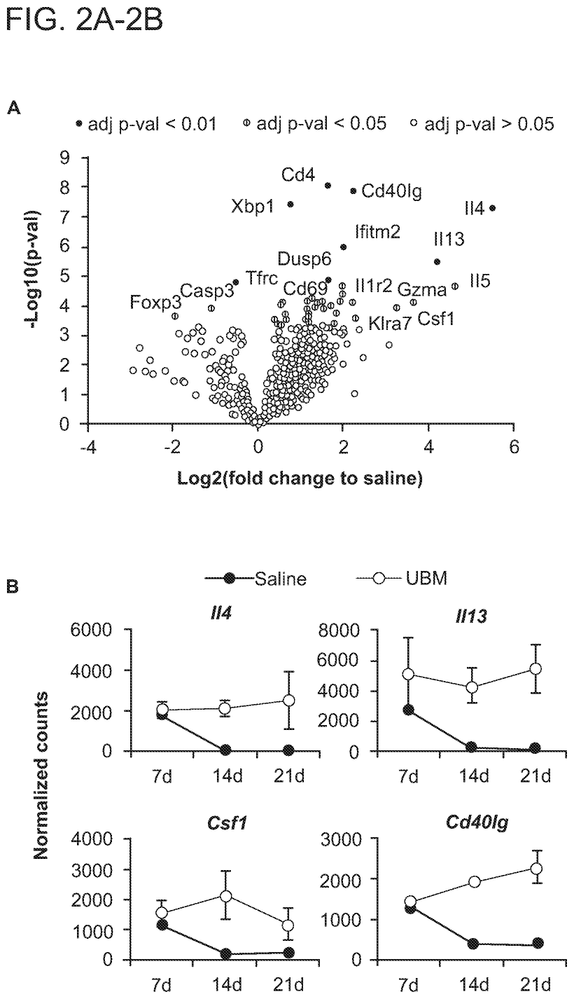

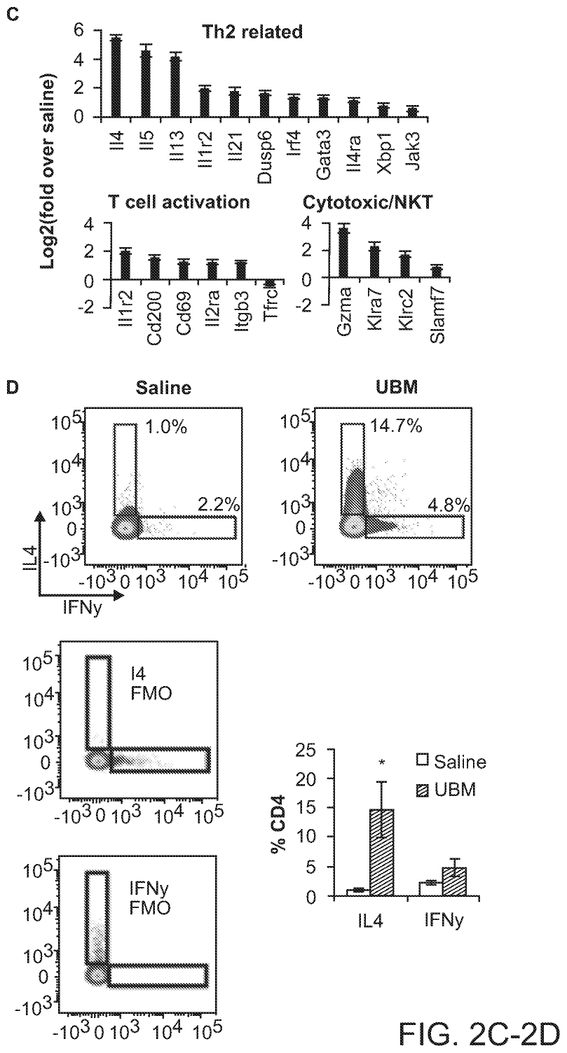

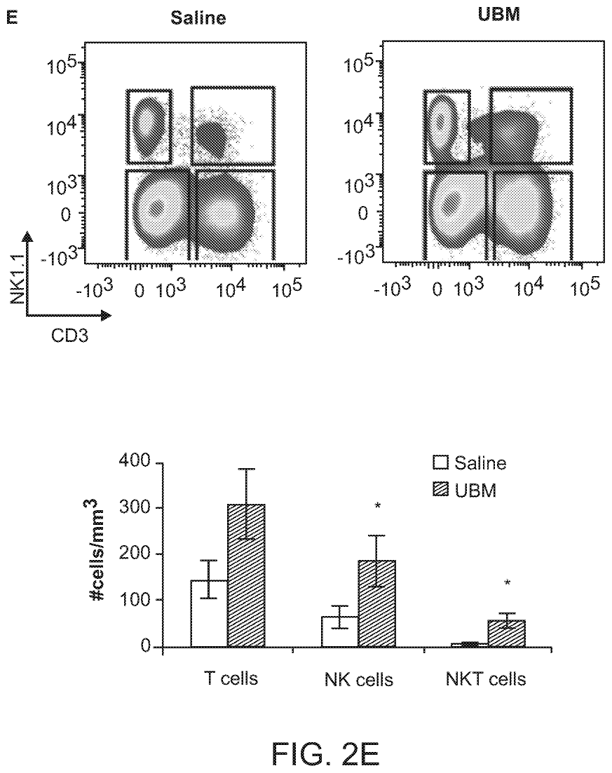

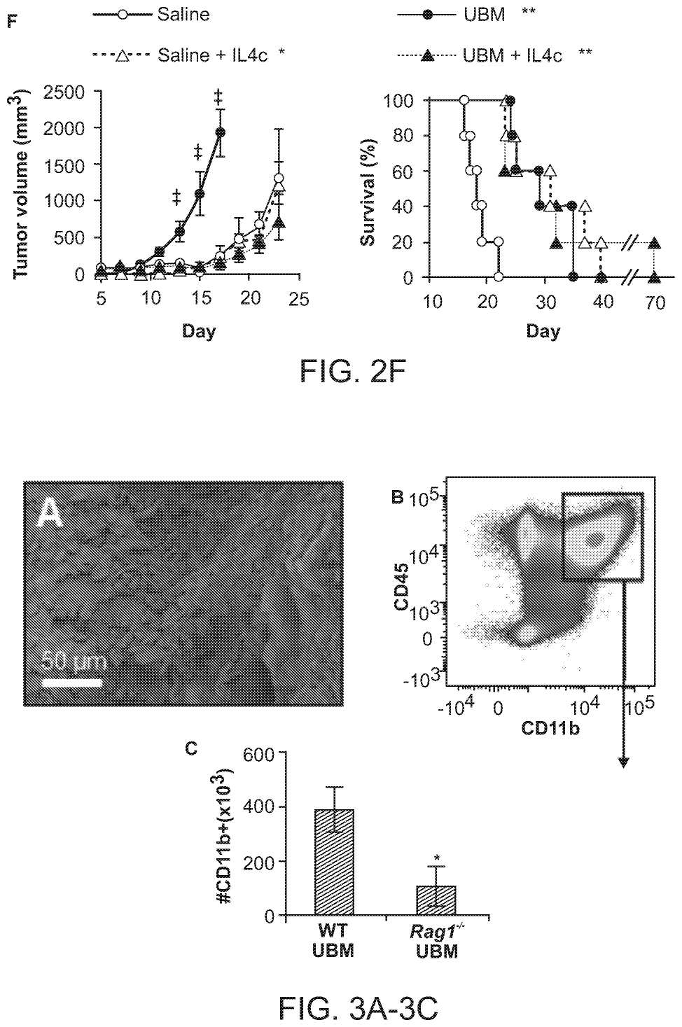

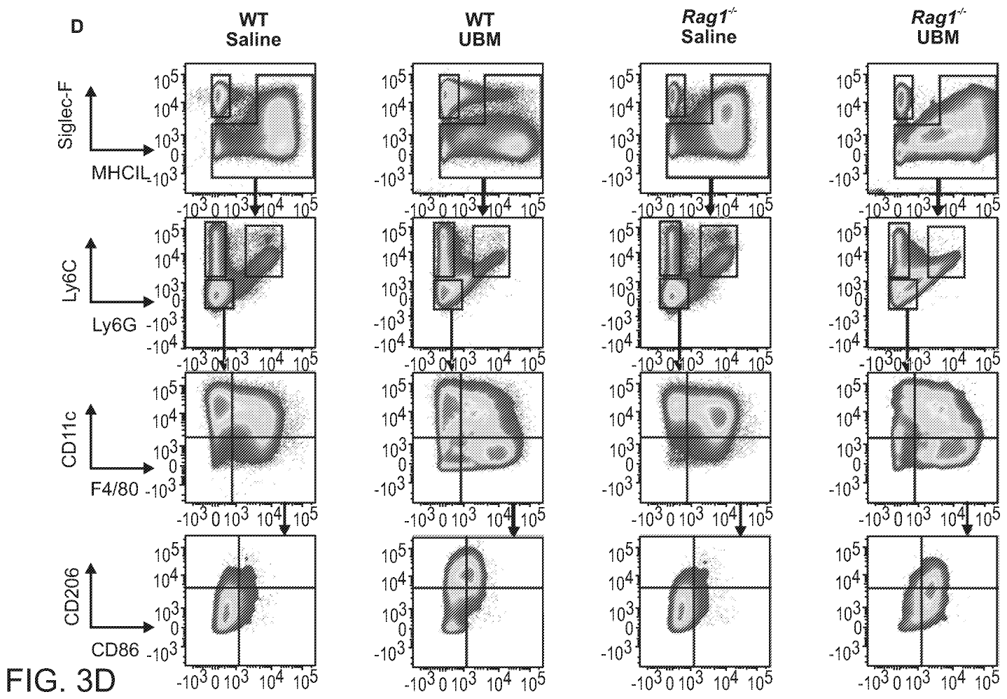

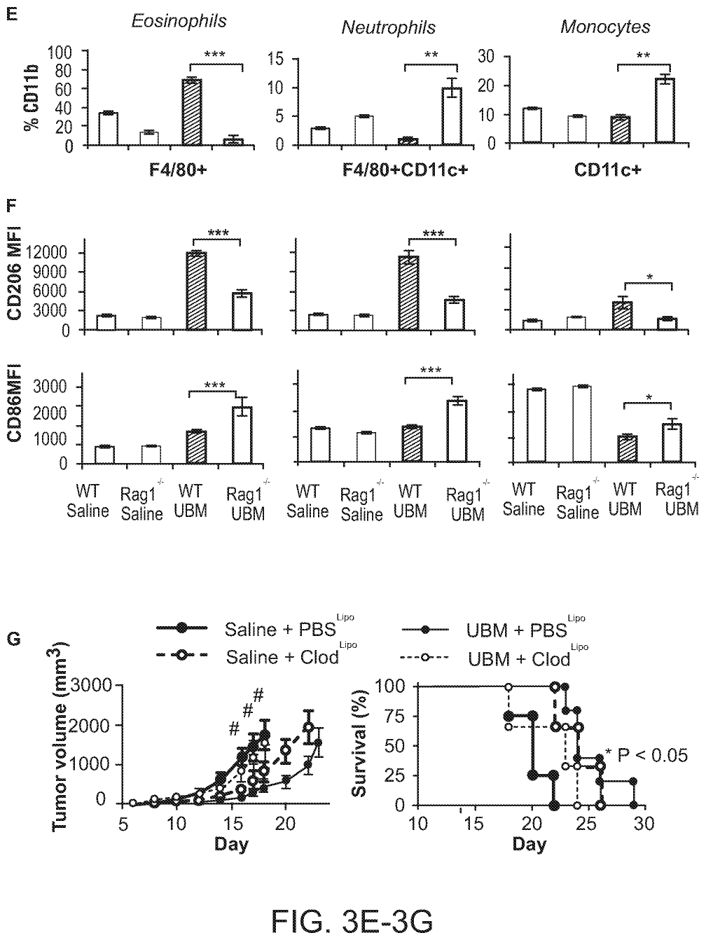

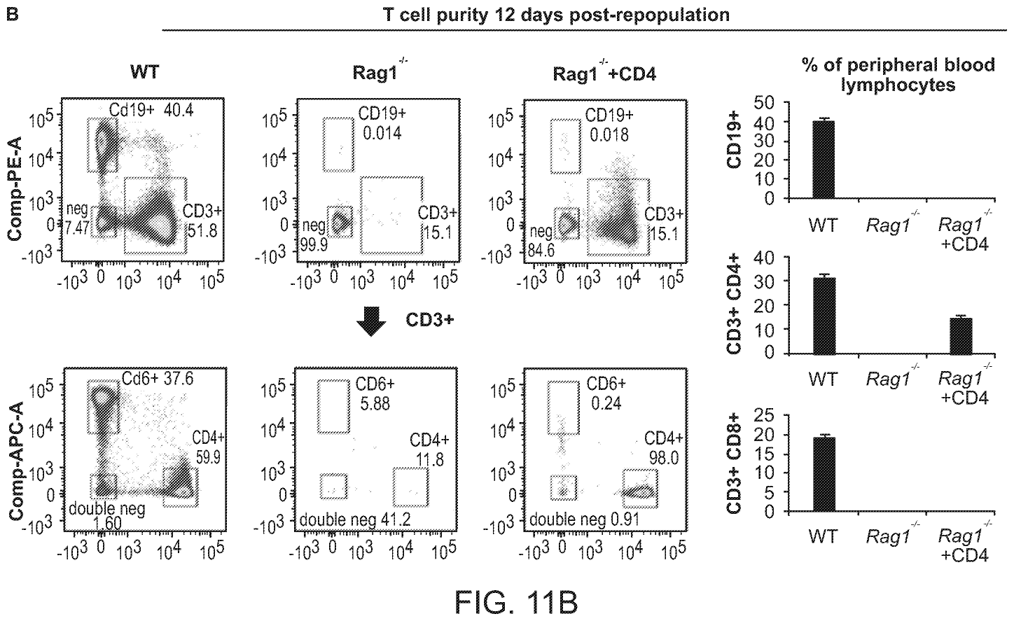

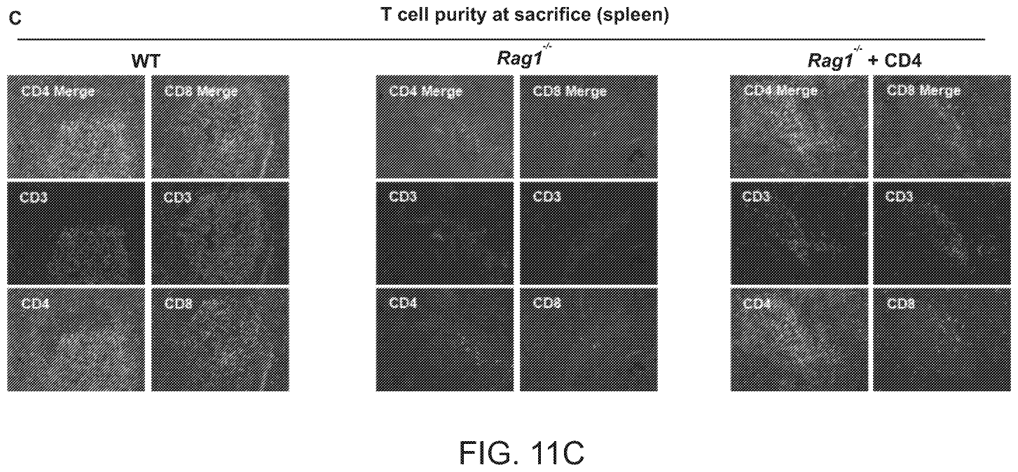

UBM associated T cells have an activated T_{H}2 phenotype compared to TILs. The UBM microenvironment depends on T cells for tumor inhibiting effects and led us to perform a detailed characterization of T cell phenotype via multiplexed gene expression analysis. WT mice were implanted with B16-F10 cells with and without UBM and harvested after 7, 14, and 21 days. T cells (CD45⁺CD3⁺F4/80⁻CD11b⁻) were sorted and gene expression analyzed for 770 immune related genes by hybridization to barcoded mRNA probes using the NanoString platform. Forty (40) genes were differentially regulated with UBM delivery compared to Saline (FIG. 2A) at 14 days. Many of the upregulated genes were consistent across time points and are known to regulate macrophage and dendritic cell activation, such as Il4, Il13, Csf1, and Cd40lg (FIG. 3B). There was a clear upregulation of T_{H}2 related genes (FIG. 2C) in UBM associated T cells relative to classical TILs obtained with Saline delivery, with the greatest fold increases occurring in the T_{H}2 cytokines Il4 (45-fold), Il15 (24-fold), and Il13 (18-fold). These T cells also showed a more activated phenotype with upregulation of activation markers (FIG. 2C) such as Cd69 (2.2-fold) and Il2ra (CD25, 2.2-fold). Consistent with flow cytometry analysis of lineage markers (FIGS. 1G, 1H), there was increased Cd4 (3-fold) and decreased Foxp3 expression (4-fold) in UBM T cells vs TILs (FIGS. 11A-11C). Furthermore, several cytotoxic associated genes were upregulated such as Gzma (Granzyme A, 12-fold), Klra7 (5-fold), and Klrc2 (3-fold) (FIG. 2C) suggesting CD3+natural killer T cell (NKT cell) involvement since CD8 cytotoxic T cells proportions were not increased with UBM.

Flow cytometry was performed to confirm the phenotypes observed with gene expression analysis. Intracellular cytokine staining and flow cytometry of UBM associated T cells after 14 days shows increased IL4 expression in CD4⁺ T cells isolated from UBM delivered tumors, validating the T_{H}2 profile determined from gene expression analysis (FIG. 2D). UBM also recruited increased densities of NK1.1⁺CD3⁻ NK cells (192.0±48.2 vs 64.8±22.3 cells/mm³) and NK1.1⁺/CD3⁺ NKT cells (58.1±15.9 vs 7.6±2 cells/mm³) compared to saline tumors (FIG. 2E). Finally, it was determined whether recruited CD4+ T cells were antigen experienced and activated via expression of CD44. The majority of CD4⁺ T cells within the tumor were antigen experienced (CD44⁺) regardless of saline or UBM delivery (FIGS. 10A-10C), however the proportion of CD44⁺CD4⁺ cells in the tumor draining lymph node increased from 15.5±1.6% with Saline delivery to 21.7±3.1% with UBM. The majority of these CD4⁺ cells were CD62L-, indicating an effector memory phenotype (FIG. 10C).

IL4 induced inflammation impairs tumor formation. It was then investigated whether the Type 2 inflammatory response to UBM could replicated, and whether this response would be inhibitory to B16-F10 tumor formation. B16-F10 cells were delivered with the canonical T_{H}2/M2 agonist IL4 in the form of a half-life stabilized IL4 complex (IL4c). IL4c with Saline greatly impaired tumor formation, increasing survival by 8 days, which was similar to UBM that increased survival by 11 days (FIG. 2F). The addition of IL4c with UBM, however, did not produce an additive survival benefit (FIG. 2F).

UBM alters myeloid cell recruitment during B16-F10 tumor formation and is lymphocyte dependent. Previous studies have shown that site appropriate tissue remodeling by ECM scaffolds (including UBM) is accompanied by robust recruitment of myeloid cells. Indeed, subcutaneous injection of acellular UBM scaffolds (without B16-F10 cells) in WT mice leads to rapid infiltration by host cells as shown by SEM (FIG. 3A), many of which are CD11b⁺ myeloid cells (FIG. 3B,C). Therefore, the myeloid cell compartment was evaluated in both WT and lymphocyte free Rag1^{-/-} mice. Myeloid cells (CD45⁺CD11b⁺) are the dominant cell type after 7 days post B16-F10 delivery with UBM. However, CD11b⁺ myeloid cell recruitment to UBM is impaired in Rag1^{-/-} mice (112,000±66,000 cells) compared to WT (391,000±191,000). Additional CD11b⁺ myeloid phenotyping revealed that the UBM immune microenvironment altered the composition of the myeloid compartment, which depended greatly on the presence of lymphocytes (FIG. 3D). Of the CD11b⁺ cells recruited by UBM in WT mice, 69.0±1.5% are Siglec-F⁺ eosinophils after 7 days, which corresponds to over double the proportion found with Saline delivery after 7 days (FIG. 3E). Eosinophil recruitment to UBM is dependent on lymphocytes, however, and is drastically reduced to 6.4±1.4% in Rag1^{-/-} mice. Few Ly6G⁺ granulocytes were present with UBM delivery (3.6±0.2% of CD11b cells), though this proportion rises in Rag1^{-/-} mice (11.0±0.8%) with Saline delivery showing a similar decrease compared to WT (FIG. 3E). The Ly6C⁺ monocyte population follows a similar trend to neutrophils (FIG. 3E).

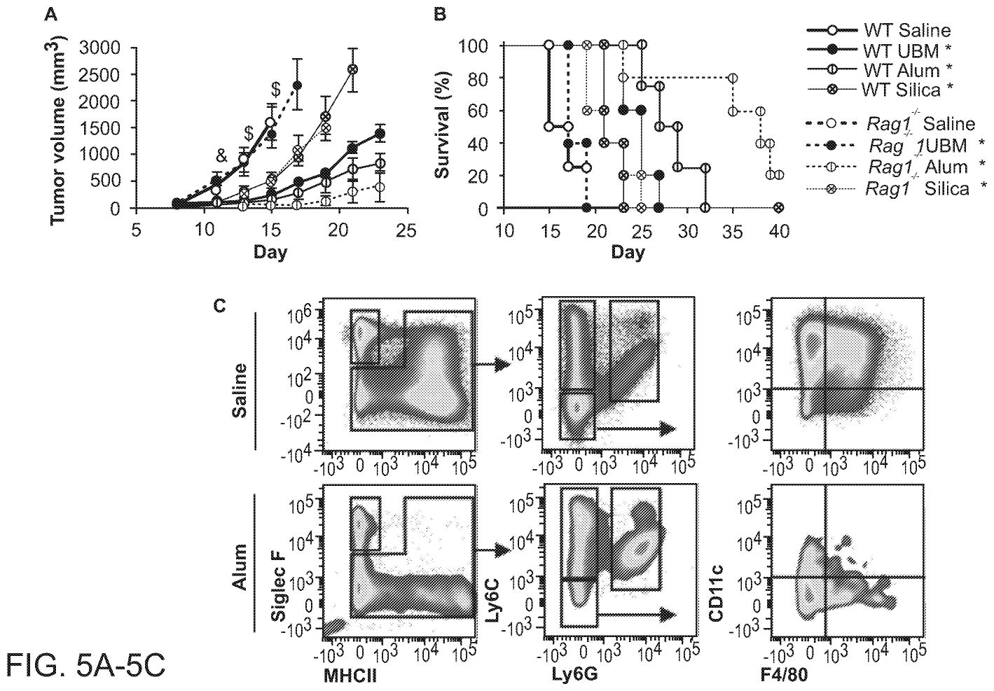

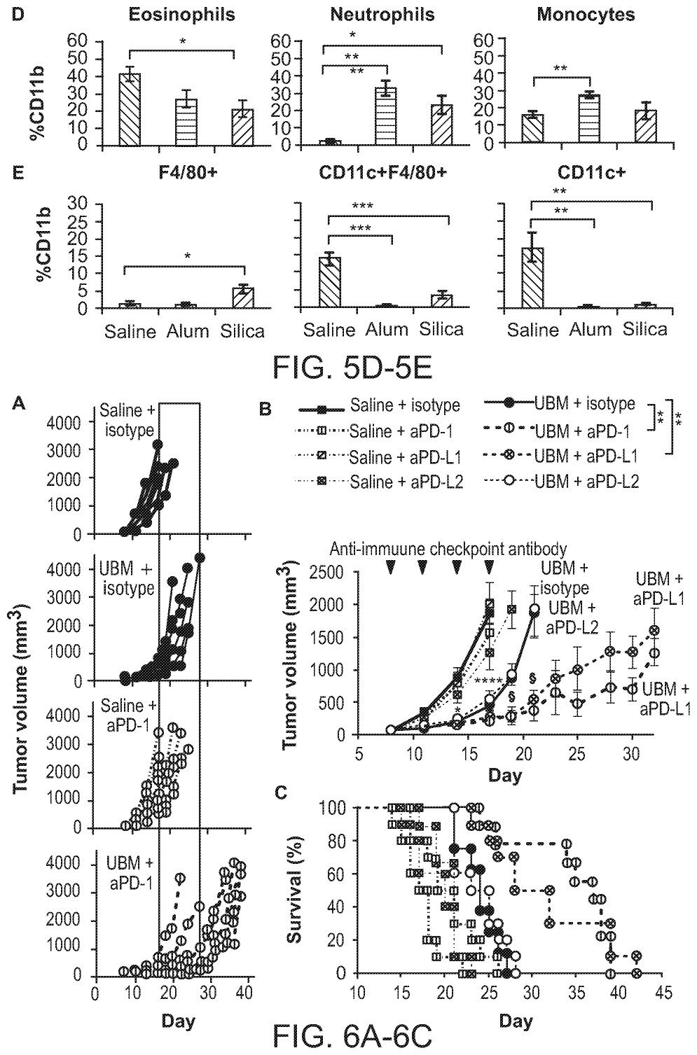

The non-monocytic/granulocytic myeloid population (Ly6C-Ly6G-) after 7 days was further characterized for macrophage polarization markers in both WT and Rag1^{-/-} mice. CD11b⁺ macrophage and dendritic cell subpopulations were analyzed for expression of the M2 associated marker CD206 and the M1 associated marker CD86. It was found that UBM implantation recruited a prevalent F4/80⁺ macrophage population with an M2 polarization bias (FIGS. 3D, 3F). Expression of the M2 associated surface marker CD206 was greatly increased in F4/80⁺ UBM associated macrophages, and this expression was greatly reduced in Rag1^{-/-} mice (FIGS. 3D, 3F). A detailed analysis of each of the separate F4/80 and CD11c myeloid subpopulations showed particularly high CD206 in F4/80+ cells (both CD11c^{+/-}) in UBM associated macrophages. CD206 expression in F4/80⁺ cells were approximately 5-fold greater than the classical tumor associated macrophages (TAMs) that are recruited with Saline delivery (FIG. 3F). Conversely, UBM associated F4/80⁺ macrophages in Rag1^{-/-} mice had approximately 2-fold greater CD86 expression (FIG. 3F) providing evidence of an M1 like phenotype in the absence of lymphocytes. UBM F4/80 and CD11c expression profiles were notably different from saline delivered B16-F10 tumor controls. Saline groups showed a greater proportion of CD11c⁺ cells (FIG. 3D), which had greater CD86 expression than WT UBM counterparts (FIGS. 5B, 5C), notably in CD11c⁺F4/80⁻ dendritic cells.

UBM associated macrophages are necessary for the anti-tumoral UBM environment. TAMs have been characterized as having an M2-like phenotype, and are implicated in promoting tumor progression. Since it was found that UBM associated macrophages possess even greater M2 polarization biases by expression of CD206, what effect macrophage ablation would have on tumor growth was characterized. Circulating macrophage progenitors were partially depleted by injecting clodronate liposomes (Clod^{Lipo}) prior to and following B16-F10/UBM injection. Flow cytometry analysis of peripheral blood confirmed that Ly6C^{hi} monocytes decreased by 86% with clodronate liposome injection compared to PBS liposome controls. (FIG. 12). Animals treated with control PBS liposomes)(PBS^{Lipo}) showed typical tumor growth with Saline delivery and significant inhibition with UBM (FIG. 3G). Surprisingly, opposite effects were observed with clodronate liposome treatment. Macrophage depletion of classical TAMs in Saline delivered tumors slowed tumor growth whereas the tumor inhibitory effect of UBM was largely lost in the absence of macrophages. These disparate effects of clodronate depletion suggested a phenotypic difference between UBM recruited macrophages and classical TAMs resulting in tumor inhibition vs tumor promotion, respectively.

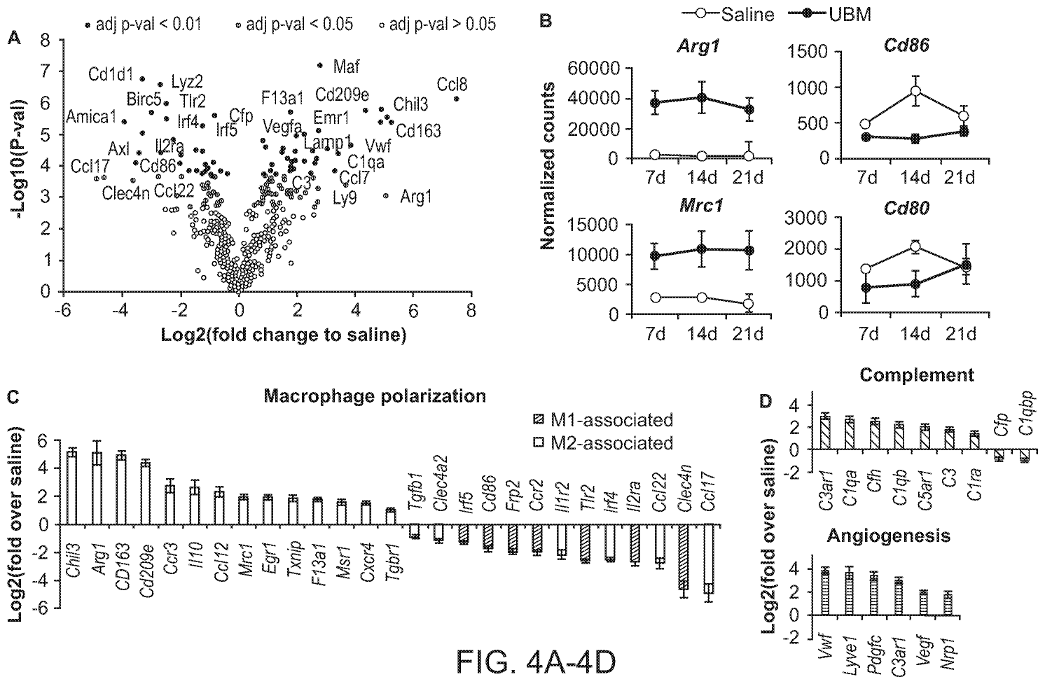

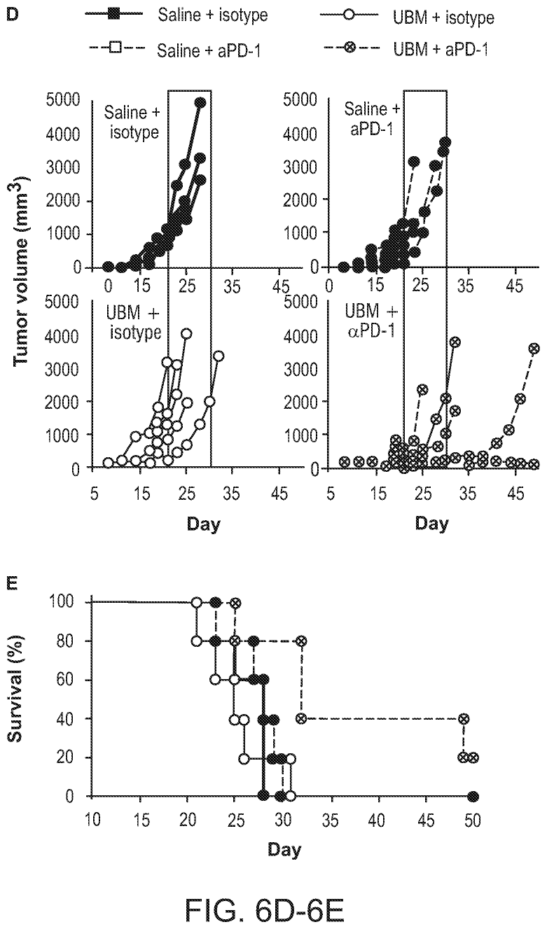

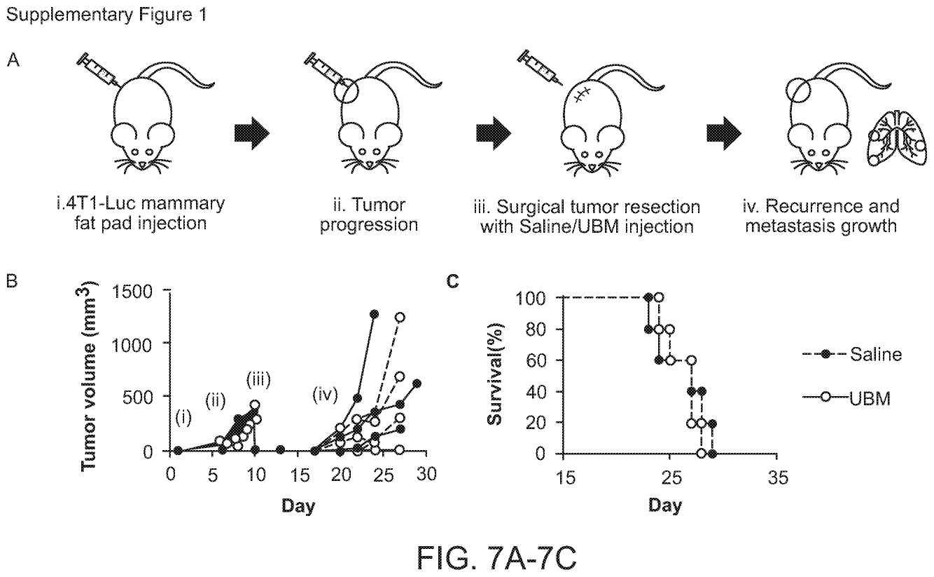

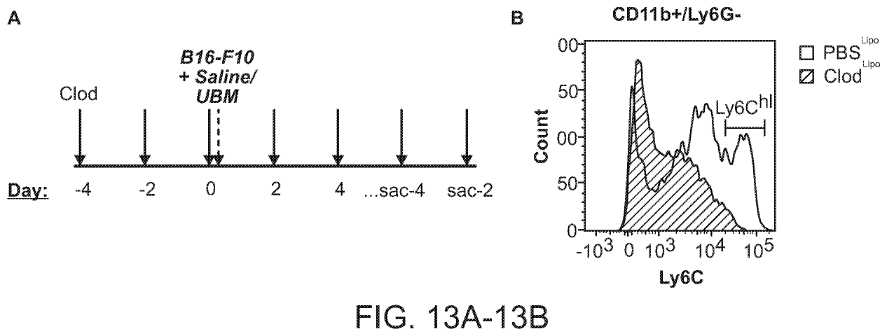

UBM associated macrophages have an increased M2 and wound healing phenotype compared to classical TAMs. Given the dependency of macrophage involvement on UBM's tumor inhibiting effects, a multiplexed gene expression analysis was performed of CD11b⁺F4/80⁺CD3⁻ cells sorted from normal saline delivered B16-F10 tumors compared to UBM delivery after 7, 14, and 21 days using the NanoString platform. Over 130 immune related genes were differentially regulated in UBM macrophages compared to classical TAMs obtained from Saline delivery (FIG. 4A) after 14 days. The largest fold changes relative to saline were increases in Ccl8 (MCP2, 181-fold), Cts1 (Cathepsin-L, 37-fold), and Chil3 (Ym1, 34-fold). UBM was associated with large increases in M2 related gene expression (such as Arg1, and Mrc1) compared to saline, and decreases in M1 related genes across all time points (FIG. 6B). Indeed, the most consistently regulated gene set was related to macrophage polarization, with most differentially regulated genes supporting a highly upregulated M2 phenotype compared to classical TAMs. Macrophages isolated from UBM after 14 days had consistently lowered expression of M1 related genes such as Cd86, Ccr2, and Il2ra (FIG. 6C). Likewise, the largest polarization gene fold changes were observed for M2 genes Chil3, Arg1 (33-fold), and Cd163 (29-fold). However, several genes generally associated with an M2 phenotype showed substantially lowered expression compared to TAMs, including the chemokines Ccl17 (29-fold decrease) and Ccl22 (7-fold decrease). Furthermore, Irf4 (5-fold decrease) expression also decreased though this is a positive regulator of several M2 genes (FIG. 6C). Genes related to classical biomaterials responses and wound healing were also examined, and found that UBM macrophages showed consistent upregulation of complement and angiogenesis related genes (FIG. 6D). Finally, macrophages showed a complex gene expression profile of genes regulating survival and differentiation (FIG. 6D), and down regulation of major histocompatibility complex (MHC) class II genes but upregulation of MHC class I genes (FIGS. 13A, 13B).

Synthetic particles impair B16-F10 tumor formation in a lymphocyte independent manner. To determine whether the tumor inhibiting microenvironment was unique to UBM or generalized to all particulate materials, this B16-F10 delivery model was applied to synthetic particles. Synthetic materials are typically used as cell and/or drug delivery vehicles, or as inflammation stimulating adjuvants rather than as direct initiators of regeneration. Aluminum hydroxide (Alum) and mesoporous silica have been well characterized as immune stimulating materials and were deliver with B16-F10 cells in WT vs Rag1^{-/-} mice for comparison to UBM (14-17). Both Alum and silica were shown to impair tumor growth in WT mice with increases in survival of 11 and 5 days respectively compared to Saline. Consistent with previous experiments, UBM slowed tumor growth and increased survival by 8 days. However, in strong contrast to UBM (in which tumor inhibition was lost in Rag1^{-/-} mice), synthetic materials were either unaffected (silica) or tumor inhibition was enhanced (Alum) in the absence of adaptive immunity (FIGS. 5A, 5B). Flow cytometry analysis of infiltrating myeloid cells after 7 days post implantation (FIG. 5C) was substantially altered from the saline delivered control B16-F10 tumor, although in a very different manner than UBM. Synthetic materials were associated with a classically inflammatory Ly6G⁺ neutrophil response (FIG. 5D), with Ly6G⁺ cells accounting for 33% and 23% of myeloid cells in Alum and Silica implants, respectively. There were also relatively few viable macrophages or dendritic cells at the site of implantation compared to saline delivered tumors or UBM, and thus macrophage polarization was not analyzed.

PD-1/PD-L1 immune checkpoint inhibition enhances the tumor inhibitory UBM microenvironment. Since it was established that a CD4 T cell dependent immune response to UBM scaffolds is inhibitory to tumor growth, it was investigated potential synergies with immune activating cancer immunotherapy. UBM was combined with immune checkpoint blockade immunotherapy targeting PD-1 (programmed cell death protein 1), PD-L1 (programmed death-ligand 1) or PD-L2 (programmed death-ligand 2). PD-1 engagement with its ligands (PD-L1 or PD-L2) provides negative feedback to T cells, and blocking these inhibitory molecules to amplify the UBM immune response. Mice began checkpoint blockade 8 days following B16-F10 implantation with Saline or UBM. Blocking either PD-1 or PD-L1 greatly slowed B16-F10 tumor growth with UBM delivery compared to isotype controls, while blocking PD-L2 had no effect (FIGS. 6A, 6B). Mean survival with UBM delivery increased from 23.8±0.5 days with isotype to 34.4±2.0 and 32±2.1 days with PD-1 and PD-L1 blockade, respectively (FIG. 6C). In contrast, checkpoint blockade did not significantly affect tumor growth with Saline delivery alone and thus the combination of PD-1/PD-L1 blockade with the UBM immune microenvironment is responsible for increased tumor inhibition. Checkpoint blockade was also examined following delayed UBM implantation to further validate the response. B16-F10 cells were inoculated into the right flanks of mice and given a day to engraft before implantation with UBM or Saline control in the same approximate area (in contrast to co-delivery where all cancer cells are in direct proximity with UBM). These mice were then treated with anti-PD-1 vs an isotype control on day 5. Although delayed UBM implantation with isotype treatment had no effect on tumor formation, PD-1 blockade with UBM impaired tumor growth (FIG. 6D) and improved survival (FIG. 6E).

### DISCUSSION

Implantable biomaterials are a foundational component of tissue engineering and regenerative medicine. Initially these materials were intended to act as “inert” scaffolds for cell and/or drug delivery, but the paradigm has shifted towards bioactive materials that interact with the host. ECM derived scaffolds from decellularized tissues have an established clinical record for this purpose. Several mechanisms are thought to play a role in the regenerative potential of implanted biologic scaffolds, though a Type 2 immune response to ECM scaffolds has been shown to be indispensable for regenerative outcomes(2-4). While strides have been made towards materials that encourage tissue healing, questions remain as to how a reparative environment affects tumor formation and whether this response can be a therapeutic. To address this concern, syngeneic cancer cell lines were delivered with UBM to study the interaction of the ECM biomaterial immune response with tumor formation. It was found that the immune environment generated by a clinically utilized ECM material did not enhance tumor growth, but rather inhibited tumor formation in a CD4 T cell dependent mechanism that could be augmented with systemic administration of PD-1/PD-L1 blocking antibodies.

ECM scaffolds are isolated by decellularizing mammalian or cadaveric tissues using chemical or mechanical means to optimize cell removal while preserving matrix composition. The logic was to provide a template with natural complexity that is beyond artificial fabrication technology. It became apparent, however, that the phenotype of immune cells recruited to ECM scaffolds dictated their ability to reconstruct tissues. Site specific tissue remodeling occurred downstream of the appropriate immune response, which has been most extensively studied with respect to M2 macrophage polarization and recently TH2 T cell polarization. Therefore, biologic ECM scaffolds can be considered immune modulating biomaterials, though it is unclear what aspects of this immune response influence tumor progression.

Wound healing shares several common molecular features with cancer (6, 18) that are a concern for regenerative medicine. For example, stem cells delivered exogenously or activated in situ, are vital for tissue replacement, but in the absence of appropriate contextual signals can become a source of neoplastic cells(19, 20). ECM scaffolds induce wound healing processes also upregulated during cancer progression such as angiogenesis, progenitor cell mobilization, and Type 2 inflammation, however these materials have not been found to promote tumor formation (11). ECM scaffolds are often used in soft tissue reconstruction, including following tumor resection, potentially in proximity to cancer cells. Despite their use in surgical oncology, the role of ECM immune responses on tumor progression has been largely overlooked. Understanding this intersection between tissue engineering and oncology may answer whether a pro-healing environment “jump starts” tumorigenesis.

The present study demonstrates that a pro-healing, Type 2-like inflammatory response induced by ECM biomaterials is compatible with tumor inhibition. This contradicts the classical view of tumoral immunity in which T_{H}1 induced M1 macrophage and cytotoxic T cell effectors are the most adept at tumor killing, while T_{H}2/M2 related cells are involved in immune suppression and tumor progression(10). Indeed, TAMs are often described as possessing an M2 expression profile that correlates with tumor growth and a poor prognosis(10, 21). However, it was found that the UBM immune microenvironment diverged from tumors in several important aspects: increased infiltration and activation of T cells, a reduction in the proportion of Tregs, increased responsiveness to immunotherapy, and an M2-like macrophage phenotype that is distinct from classical TAMs.

Several observations support the notion of a unique UBM associated macrophage phenotype that is functionally distinct from classical TAMs. The degree of expression is substantially greater in UBM macrophages compared to classical TAMs; multiple macrophage polarization markers at the transcript and protein level (e.g. CD206) are highly upregulated in UBM associated macrophages. In addition to M2 surface markers, UBM associated macrophages highly upregulated angiogenic mediators, complement genes, and numerous chemokines. This activated M2-like state is severely impaired without CD4 T cells. UBM associated macrophages shift towards an M1 phenotype in Rag1^{-/-} mice and tumors instead grow unhindered, challenging the paradigm that M1 macrophage polarization is favorable for tumor inhibition in every context. Blocking PD-L2, which is preferentially expressed on myeloid cells to suppress T cell activation, did not affect the immune environment suggesting that UBM macrophages were not inhibitory by this mechanism. Thus, a binary M1/M2 model may not suffice for predicting anti-tumoral immunity and that the level of activation must be considered. A hyperacute pro-healing M2 environment may be disruptive to tumor growth, and thus, while the “type” of inflammation is instrumental, the intensity, duration, and context cannot be overlooked.

A unique UBM macrophage population would also explain the opposing effects of macrophage depletion with clodronate. Consistent with previous studies, depletion of tumor promoting TAMs is effective at slowing tumor growth (22). In contrast, depleting UBM associated macrophages had the opposite effect and created a tumor permissive environment adjacent to the UBM material, suggesting that classical TAMs and UBM associated macrophages have opposing functions in this context. Finally, the UBM macrophage phenotype does not fit completely into known M2 archetypes. Compared to TAMs, gene expression of the M2 chemokines Ccl17 and Ccl22, which are important for regulatory Treg recruitment(23), is greatly reduced in UBM macrophages, and correlates with the observed decrease in Treg frequency. The most highly upregulated gene in UBM associated macrophages was Ccl8 (encoding CCL8/MCP-2), a potent chemotactic agent. While not traditionally associated with an M1/M2 phenotype, CCL8 has greater chemotactic activity on highly differentiated T_{H}2 cells over T_{H}1 or myeloid cells (24). This supports the strong T cell recruitment and T_{H}2 polarization observed, with T cells expressing high levels of Il4 and Il13 relative to classical TILs. Thus, these T_{H}2 cells may also represent a more active T_{H}22 phenotype than is found in classical T_{H}2 biased TILs. This pairs with evidence that activated CD4⁺ T cells can be effective at tumor killing indirectly via instruction of myeloid cells and NK cells (25).

Tumor inhibition and the UBM associated macrophage phenotype was clearly dependent on CD4 T helper cells. In the complete absence of CD4⁺ T cells, UBM becomes an excellent substrate for tumor growth, which also confirms that UBM is not directly repressing tumor formation by toxic or physical means. A likely interpretation is that increased T cell recruitment is a way to improve immune recognition of otherwise poorly immunogenic tumors like B16-F10 melanoma. It was further evaluated whether the UBM T cell response promoted tumor inhibition (rather than the T_{H}2 response opposing another mechanism of inhibition) by application of checkpoint immunotherapy. Blocking immune checkpoint molecules such as PD-1 and PD-L1 may prevent exhaustion, anergy, and/or apoptosis of UBM infiltrating T cells thereby amplifying the immune response. It was found that PD-1/PD-L1 blockade substantially enhanced tumor inhibition only in combination with UBM thus supporting a tumor inhibitory environment. This also demonstrates compatibility of ECM scaffolds for use as a therapeutic in cancerous environments in conjunction with immunotherapy. M2 and T_{H}2 polarization was induced by UBM implantation. It was found that injection of IL4c (a canonical T_{H}2/M2 mediator) with B16-F10 cells also resulted in reduced tumor growth supporting that IL4 may be an effector. Several studies investigating IL4 secreting cancer cell vaccines have shown that IL4 driven inflammation promotes local cancer rejection and anti-tumor immune memory (26, 27). The UBM may act similarly, but stimulation IL4 production from endogenous sources.

Biomaterials scaffolds have previously been used as tools in cancer immunoengineering. Synthetic material scaffolds and particles have been implanted to improve cancer vaccines by inherent immune stimulating adjuvant activity and/or as drug delivery vehicles (17, 28, 29). Comparing UBM to synthetic particulate adjuvants such as Alum and Silica offers insights into the disparity between synthetic and biologic materials. Like UBM, Alum is also characterized as efficiently promoting a T_{H}2 response (30) via several proposed mechanisms: inflammasome activation, direct membrane lipid binding, and local cell damage/death at the site of implantation (14-16). However, unlike UBM, local immune infiltrates in Alum and Silica were granulocyte dominant, which is consistent with classical destructive inflammation rather than the M2-like macrophage UBM response. Local tumor inhibition was observed with Alum or Silica particle delivery, though inhibition also occurred in the absence of lymphocytes. Thus, while both UBM and synthetic materials delay tumors, they act via differing mechanisms: synthetic particles activating cytotoxic innate responses and UBM requiring regulation by CD4 T cells. Once tumor nodules had formed around UBM or synthetic particles, however, tumor growth rate normalized. It is possible that once the tumor microenvironment had been established, it insulates the cancer from the local UBM induced immune response. It is also possible that the intensity of the immune response diminishes over time. This timeline is consistent with previous studies on the kinetics of material immune responses that show peak foreign body immune responses at 7-14 days.

Several potential effectors of UBM induced tumor inhibition were revealed in this study. Increased frequencies of macrophages, eosinophils, NK cells, NKT cells, or a decrease in Tregs are potential candidate mechanisms. Increased complement and complement receptor gene expression by macrophages, was also found which has been correlated to tumor rejection (31). Many clinical ECM products, including the UBM used in the present study, are of xenogeneic tissue origin. The baseline antigen in UBM does not lead to implant rejection as shown by a wealth of clinical and pre-clinical data, likely due to removal of the majority of cell components ((including MHC class I (12)) and the highly conserved nature of ECM proteins. Therefore, it is possible that low-level xenogeneic antigen presentation and recognition is responsible for the observed T_{H}2 responses, and that introduction of T_{H}2 stimulating antigens may provide non-destructive protection from tumor growth.

This study shows that ECM materials used in tissue reconstruction are compatible in environments where cancer may occur. UBM material induced immune responses include a unique M2-like macrophage population dissimilar from classical TAMs, which requires CD4⁺ cells with an activated T_{H}2 profile. This work provides insights on biomaterials based methods of manipulating the tumor microenvironment through using alternative inflammatory activation and further regulation by checkpoint blockade immunotherapy.

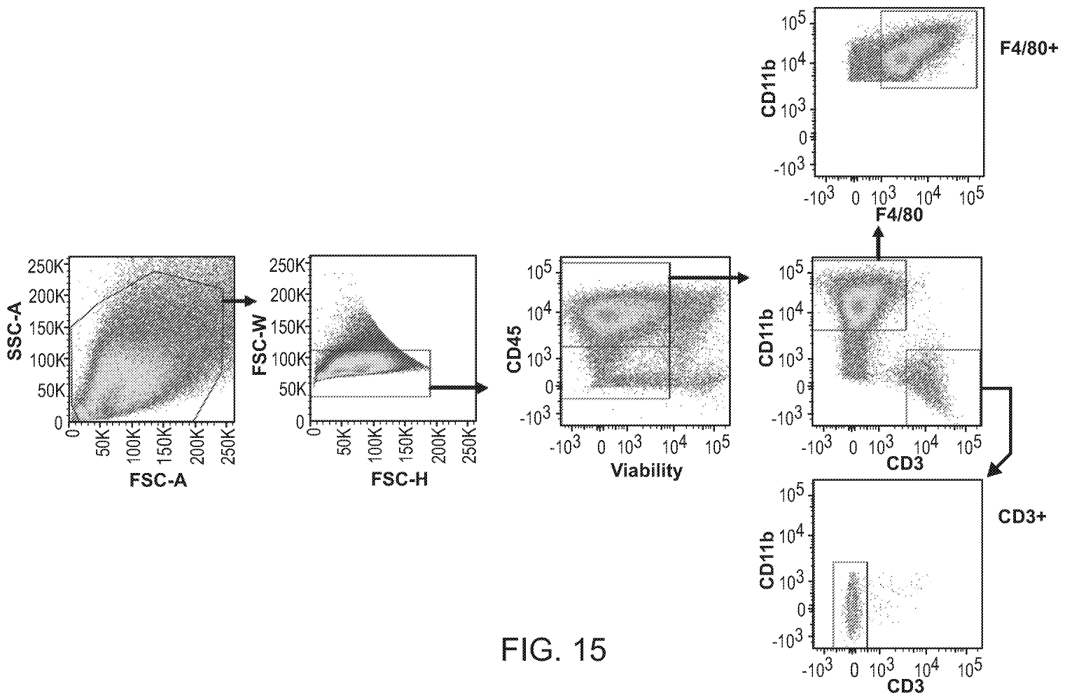

TABLE 1
Antibodies used in flow cytometry experiments.

| Marker | Conjugate | Clone | Dilution | Manufacturer | Catalog # | FIG. ref. |
|---|---|---|---|---|---|---|
| Viability | eFluor780 | — | 1:1000 | ThermoFisher | 65-0865- | FIGS. |
| | | | | | 14 | 3B, D, 5C, |
| | | | | | | 15 |
| Viability | Aqua | — | 1:1000 | ThermoFisher | L34957 | FIGS. 1H, |
| | | | | | | 2D, 2E, |
| | | | | | | 10A, 10B, |
| | | | | | | 11A, 11B |
| CD45 | BV605 | 30-F11 | 1:100 | Biolegend | 103139 | FIGS. 3B, |
| | | | | | | 3D, 5C, 15 |
| CD45 | PerCP-Cy5.5 | 30-F11 | 1:100 | Biolegend | 103131 | FIGS. 1H, |
| | | | | | | 2D, 2E, |
| | | | | | | SFig 10A, |
| | | | | | | 10B, 11A, |
| | | | | | | 11B |
| 2MHC II (I- | AF488 | M5/114 | 1:200 | Biolegend | 107615 | FIGS. 3B, |
| A/I-E) | | | | | | 3D, 5C |
| CD11b | AF700 | M1/70 | 1:300 | Biolegend | 101222 | FIGS. 3B, |
| | | | | | | 3D, 5C, 15 |
| Siglec-F | PE-CF594 | E50-2440 | 1:200 | BD Biosciences | 562757 | FIGS. 3B, |
| | | | | | | 3D, 5C |
| Ly6C | PerCP-Cy5.5 | HK1.4 | 1:400 | Biolegend | 128011 | FIGS. 3B, |
| | | | | | | 3D, 5C |
| Ly6G | Pacific Blue | 1A8 | 1:400 | Biolegend | 127611 | FIGS. 3B, |
| | | | | | | 3D, 5C |
| F4/80 | PE-Cy7 | BM8 | 1:250 | Biolegend | 123113 | FIGS. 3B, |
| | | | | | | 3D, 5C, 15 |
| CD11c | APC | N418 | 1:250 | Biolegend | 117309 | FIGS. 3B, |
| | | | | | | 3D, 5C |
| CD206 | PE | C068C2 | 1:250 | Biolegend | 141705 | FIGS. 3B, |
| | | | | | | 3D, 5C |
| CD86 | BV510 | GL-1 | 1:200 | Biolegend | 105039 | FIGS. 3B, |
| | | | | | | 3D, 5C |
| CD3 | AF488 | 17A2 | 1:150 | Biolegend | 100212 | FIGS. 1H, |
| | | | | | | 2D, 1E, |
| | | | | | | 11A, 11B |
| CD3 | APC | 17A2 | 1:150 | Biolegend | 100235 | FIG. 15 |
| CD4 | PE-Cy7 | GK1.5 | 1:300 | Biolegend | 100422 | FIGS. 1H, |
| | | | | | | 2D, 2E, |
| | | | | | | 10A, 10B, |
| | | | | | | 11A, 11B |
| CD8 | AF700 | 53-6.7 | 1:200 | Biolegend | 100729 | FIGS. 1H, |
| | | | | | | 2D, 2E, |
| | | | | | | 10A, 10B, |
| | | | | | | 11A, 11B |
| FoxP3 | Pacific Blue | MF-14 | 1:150 | Biolegend | 126409 | FIGS. 1H, |
| | | | | | | 2D |
| IL4 | PE | 11B11 | 1:150 | Biolegend | 504103 | FIG. 2D |
| IFNγ | BV605 | XMG1.2 | 1:150 | Biolegend | 505839 | FIG. 2D |
| NK1.1 | APC | PK136 | 1:400 | Biolegend | 108709 | FIGS. 2E, |
| | | | | | | 10A, 10B |
| CD44 | BV605 | IM7 | 1:200 | Biolegend | 103047 | FIGS. 10A- |
| | | | | | | 10C |
| CD62L | APC-Cy7 | MEL-14 | 1:200 | Biolegend | 104427 | FIGS. 10A- |
| | | | | | | 10C |
| CD19 | PE | 6D5 | 1:400 | Biolegend | 115507 | FIGS. 10A- |
| | | | | | | 10C 11A, |
| | | | | | | 11B |
| CD16/32 | — | 9 | 1:50 | Biolegend | 101302 | All panels |

### REFERENCES

1. S. F. Badylak, D. O. Freytes, T. W. Gilbert, Extracellular matrix as a biological scaffold material: Structure and function. Acta Biomater 5, 1-13 (2009).
2. A. J. Allman et al., Xenogeneic extracellular matrix grafts elicit a TH2-restricted immune response. Transplantation 71, 1631-1640 (2001).
3. S. F. Badylak, J. E. Valentin, A. K. Ravindra, G. P. McCabe, A. M. Stewart-Akers, Macrophage phenotype as a determinant of biologic scaffold remodeling. Tissue Eng Part A 14, 1835-1842 (2008).
4. K. Sadtler et al., Developing a pro-regenerative biomaterial scaffold microenvironment requires T helper 2 cells. Science 352, 366-370 (2016).
5. J. M. Anderson, A. Rodriguez, D. T. Chang, Foreign body reaction to biomaterials. Semin Immunol 20, 86-100 (2008).
6. H. F. Dvorak, Tumors: wounds that do not heal-redux. Cancer Immunol Res 3, 1-11 (2015).
7. M. R. Junttila, F. J. de Sauvage, Influence of tumour micro-environment heterogeneity on therapeutic response. Nature 501, 346-354 (2013).
8. I. Mellman, G. Coukos, G. Dranoff, Cancer immunotherapy comes of age. Nature 480, 480-489 (2011).
9. K. Sadtler et al., The Scaffold Immune Microenvironment: Biomaterial-Mediated Immune Polarization in Traumatic and Nontraumatic Applications. Tissue Eng Part A, (2016).
10. D. G. DeNardo et al., CD4(+) T cells regulate pulmonary metastasis of mammary carcinomas by enhancing protumor properties of macrophages. Cancer Cell 16, 91-102 (2009).
11. J. P. Hodde, M. A. Suckow, W. R. Wolter, M. C. Hiles, Small intestinal submucosa does not promote PAIII tumor growth in Lobund-Wistar rats. J Surg Res 120, 189-194 (2004).
12. K. Sadtler et al., Proteomic composition and immunomodulatory properties of urinary bladder matrix scaffolds in homeostasis and injury. Semin Immunol, (2017).
13. B. Brown, K. Lindberg, J. Reing, D. B. Stolz, S. F. Badylak, The basement membrane component of biologic scaffolds derived from extracellular matrix. Tissue Eng 12, 519-526 (2006).
14. T. L. Flach et al., Alum interaction with dendritic cell membrane lipids is essential for its adjuvanticity. Nat Med 17, 479-487 (2011).
15. V. Hornung et al., Silica crystals and aluminum salts activate the NALP3 inflammasome through phagosomal destabilization. Nat Immunol 9, 847-856 (2008).
16. T. Marichal et al., DNA released from dying host cells mediates aluminum adjuvant activity. Nat Med 17, 996-1002 (2011).
17. J. Kim et al., Injectable, spontaneously assembling, inorganic scaffolds modulate immune cells in vivo and increase vaccine efficacy. Nat Biotechnol 33, 64-72 (2015).
18. H. F. Dvorak, Tumors: wounds that do not heal. Similarities between tumor stroma generation and wound healing. N Engl J Med 315, 1650-1659 (1986).
19. Y. Ge et al., Stem Cell Lineage Infidelity Drives Wound Repair and Cancer. Cell 169, 636-650 e614 (2017).
20. B. Blum, O. Bar-Nur, T. Golan-Lev, N. Benvenisty, The anti-apoptotic gene survivin contributes to teratoma formation by human embryonic stem cells. Nat Biotechnol 27, 281-287 (2009).
21. X. Yuan et al., Prognostic significance of tumor-associated macrophages in ovarian cancer: A meta-analysis. Gynecol Oncol 147, 181-187 (2017).
22. A. Mantovani, F. Marchesi, A. Malesci, L. Laghi, P. Allavena, Tumour-associated macrophages as treatment targets in oncology. Nat Rev Clin Oncol 14, 399-416 (2017).
23. Y. Mizukami et al., CCL17 and CCL22 chemokines within tumor microenvironment are related to accumulation of Foxp3+ regulatory T cells in gastric cancer. Int J Cancer 122, 2286-2293 (2008).
24. S. A. Islam et al., Mouse CCL8, a CCR8 agonist, promotes atopic dermatitis by recruiting IL-5+T(H)2 cells. Nat Immunol 12, 167-177 (2011).
25. A. Perez-Diez et al., CD4 cells can be more efficient at tumor rejection than CD8 cells. Blood 109, 5346-5354 (2007).
26. P. T. Golumbek et al., Treatment of established renal cancer by tumor cells engineered to secrete interleukin-4. Science 254, 713-716 (1991).
27. R. I. Tepper, P. K. Pattengale, P. Leder, Murine interleukin-4 displays potent anti-tumor activity in vivo. Cell 57, 503-512 (1989).
28. S. A. Bencherif et al., Injectable cryogel-based whole-cell cancer vaccines. Nat Commun 6, 7556 (2015).
29. A. Z. Wang, R. Langer, O. C. Farokhzad, Nanoparticle delivery of cancer drugs. Annu Rev Med 63, 185-198 (2012).
30. J. L. Grun, P. H. Maurer, Different T helper cell subsets elicited in mice utilizing two different adjuvant vehicles: the role of endogenous interleukin 1 in proliferative responses. Cell Immunol 121, 134-145 (1989).
31. J. C. Doloff, D. J. Waxman, Transcriptional profiling provides insights into metronomic cyclophosphamide-activated, innate immune-dependent regression of brain tumor xenografts. BMC Cancer 15, 375 (2015).
32. V. Z. Beachley et al., Tissue matrix arrays for high-throughput screening and systems analysis of cell function. Nat Methods 12, 1197-1204 (2015).

### Other Embodiments

From the foregoing description, it will be apparent that variations and modifications may be made to the invention described herein to adopt it to various usages and conditions. Such embodiments are also within the scope of the following claims.

1. A method of preventing or treating cancer in a subject, comprising administering to the subject a biocompatible scaffold, wherein the biocompatible scaffold recruits myeloid and lymphoid cells.

2. The method of claim 1, wherein the biocompatible scaffold is implantable in a subject.

3. The method of claim 1, wherein the biocompatible scaffold is pro-regenerative.

4. The method of claim 1, wherein the biocompatible scaffold comprises a biocompatible synthetic material(s), a biomaterial(s) or combinations thereof.

5. The method of claim 1, wherein the biocompatible scaffold comprises an extracellular matrix.

6. The method of claim 5, wherein the biocompatible scaffold is a urinary bladder matrix (UBM) scaffold.

7. The method of claim 1, wherein the biocompatible scaffold further comprises one or more immune cell modulating agents.

8. The method of claim 7, wherein the one or more immune cell modulating agents are administered to the subject.

9. The method of claim 7, wherein the immune cell modulating agents comprise: cytokines, monokines, chemokines, checkpoint agents, adjuvants, vaccines, antigens, chemotherapeutic agents or combinations thereof.

10. The method of claim 9, wherein the checkpoint agent is an inhibitor of programmed death-ligand 1 (PD-L1), programmed cell death protein 1 (PD-1) and/or CTLA4.

11. The method of claim 1, wherein the biocompatible scaffold optionally comprises tumor cells, tumor cell membranes, tumor cell fragments or combinations thereof.

12. The method of claim 11, wherein the tumor cells are replication deficient.

13. (canceled)

14. The method of claim 1, further comprising administering to the subject CD4⁺ T cells.

15. The method of claim 14, wherein the CD4⁺ T cells are autologous, haploidentical, or combinations thereof.

16. The method of claim 1, optionally comprising administering to the subject stem cells, chimeric antigen T (CAR-T) cells, CAR natural killer cells (CAR-NK), bone marrow cells or combinations thereof.

17. A method of inducing adaptive immunity in a subject in need thereof, comprising administering to the subject a biocompatible scaffold, wherein the biocompatible scaffold comprises a biocompatible synthetic material, biomaterial or combinations thereof.

18-22. (canceled)

23. A biocompatible scaffold, wherein the biocompatible scaffold comprises a biocompatible synthetic material, a biomaterial, an extracellular matrix or combinations thereof.

24. The biocompatible scaffold of claim 24, wherein the extracellular matrix is a urinary bladder matrix (UBM).

25-29. (canceled)

30. A vaccine comprising micronized tissues and at least one of soluble tumor cell antigens, membrane-bound tumor antigens, replication deficient tumor cells, tumor cell tissue fragments or combinations thereof.

31. The vaccine of claim 30, wherein the micronized tissues are decellularized.

32-39. (canceled)
